(19) United States
(12) Patent Application Publication
(10) Pub. No.: US 2016/0208329 A1
(43) Pub. Date: Jul. 21, 2016

(54) SYSTEMS, DEVICES AND METHODS FOR ANTI-TL1A THERAPY

(71) Applicant: CEDARS-SINAI MEDICAL CENTER, Los Angeles, CA (US)

(72) Inventors: Stephan R. Targan, Santa Monica, CA (US); Rebecca Gonsky, Los Angeles, CA (US); Richard Deem, Azusa, CA (US)

(73) Assignee: Cedars-Sinai Medical Center, Los Angeles, CA (US)

(21) Appl. No.: 14/915,544

(22) PCT Filed: Sep. 5, 2014
(86) PCT No.: PCT/US2014/054425

§ 371 Date: Feb. 29, 2016

Related U.S. Application Data
(60) Provisional application No. 61/874,487, filed on Sep. 6, 2013.

(51) Int. Cl. C12Q 1/68

(52) U.S. Cl. CPC C12Q 1/6883; C12Q 2600/158; C12Q 2600/16; C12Q 2600/106

(57) ABSTRACT

The present invention relates to biomarker genes for diagnosing and treating diseases. Provided herein are systems and methods of diagnosing a disease in a patient based on the patient's expression levels of biomarker genes. Examples of the TL1A-associated disease include, but are not limited to, an inflammatory bowel disease (IBD), Crohn's disease (CD), ulcerative colitis (UC), and fibrosis. Also provided herein are systems and methods of identifying a patient likely to be responsive to an anti-TL1A therapy, prescribing and/or administrating an anti-TL1A therapy to the patient based on the patient's expression levels of biomarker genes.

### FIELD OF INVENTION

This invention relates to diagnosis and treatment of inflammatory and immune diseases. More specifically, this invention relates to systems, devices and methods for diagnosing and treating a disease that is susceptible to an anti-TL1A therapy.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

TL1A activation is involved in pathogenesis of a variety of inflammatory and immune diseases. For example, genome-wide association studies (GWAS) have implicated TL1A in the pathogenesis of inflammatory bowel disease (IBD) such as Crohn's disease (CD). Evidence in preclinical mouse models also supports the role of TL1A in the pathogenesis of IBD. In addition, intestinal tissues from CD patients demonstrate increased expression of TL1A at sites of active disease. However, IBD is a heterogeneous disease, and previously, treatment of IBD patients has been by trial and error. While anti-TL1A therapy (e.g., treatment with an anti-TL1A antibody) is of help to some CD patients, not all patients will benefit from anti-TL1A therapy.

As such, there is a need of biomarkers, devices, systems, and methods for defining a biomarker signature for TL1A activation, for identifying patients who have most likely been inflicted by TL1A activation and will be most suitable for anti-TL1A therapy, and for guiding treatment options for these patients.

### SUMMARY OF THE INVENTION

Various embodiments of the present invention provide a method of selecting a treatment for a subject. The method may comprise or may consist essentially of or may consist of: obtaining a sample from the subject, assaying the expression level of one or more biomarkers associated with TL1A signaling in the sample, comparing the expression level to a reference value of expression level, and prescribing an anti-TL1A therapy to the subject if the subject has a high expression level relative to the reference value, or prescribing no anti-TL1A therapy to the subject if the subject does not have a high expression level relative to the reference value.

Various embodiments of the present invention provide a method of identifying a subject likely to be responsive to an anti-TL1A therapy. The method may comprise or may consist essentially of or may consist of: obtaining a sample from the subject, assaying the expression level of one or more biomarkers associated with TL1A signaling in the sample, comparing the expression level to a reference value of expression level, and identifying the subject as likely to be responsive to an anti-TL1A therapy if the subject has a high expression level relative to the reference value, or identifying the subject as unlikely to be responsive to an anti-TL1A therapy if the subject does not have a high expression level relative to the reference value.

Various embodiments of the present invention provide a method of treating a subject with an anti-TL1A therapy. The method may comprise or may consist essentially of or may consist of: obtaining a sample from the subject, assaying the expression level of one or more biomarkers associated with TL1A signaling in the sample, comparing the expression level to a reference value of expression level, and administering an anti-TL1A therapy to the subject if the subject has a high expression level relative to the reference value, or administering no anti-TL1A therapy to the subject if the subject does not have a high expression level relative to the reference value.

Various embodiments of the present invention provide a method of diagnosing a disease in a subject. The method may comprise or may consist essentially of or may consist of: obtaining a sample from the subject, assaying the expression level of one or more biomarkers associated with TL1A signaling in the sample, comparing the expression level to a reference value of expression level, and diagnosing a TL1A-associated disease in the subject if the subject has a high expression level relative to the reference value, or diagnosing no TL1A-associated disease in the subject if the subject does not have a high expression level relative to the reference value.

Various embodiments of the present invention provide a method diagnosing susceptibility to a TL1A-associated disease in a subject. The method may comprise or may consist essentially of or may consist of: obtaining a sample from the subject, assaying the expression level of one or more biomarkers associated with TL1A signaling in the sample, comparing the expression level to a reference value of expression level, and diagnosing susceptibility to a TL1A-associated disease in the subject if the subject has a high expression level relative to the reference value, or diagnosing no susceptibility to a TL1A-associated disease in the subject if the subject does not have a high expression level relative to the reference value.

Various embodiments of the present invention provide a method of treating a disease in a subject. The method may comprise or may consist essentially of or may consist of: administering an anti-TL1A therapy to the subject, thereby treating the disease, wherein the subject has a high expression level relative to a reference value of one or more biomarkers associated with TL1A signaling.

Various embodiments of the present invention provide a method for diagnosing a disease in a subject. The method may comprise or may consist essentially of or may consist of: obtaining a sample from a subject; assaying the expression level of one or more genes in the sample; comparing the expression level to a reference value of expression level of the one or more genes; and diagnosing a disease in the subject according to the relative difference between the expression level and the reference value. In some embodiments, the method further comprises diagnosing the disease in the subject if the subject has an expression level higher than the reference value, or not diagnosing the disease in the subject if the subject does not have an expression level higher than the reference value. In other embodiments, the method further comprises diagnosing the disease in the subject if the subject has an expression level lower than the reference value, or not diagnosing the disease in the subject if the subject does not have an expression level lower than the reference value. In various further embodiments, the method further comprises prescribing an anti-TL1A therapy to the subject if the subject is diagnosed with the disease. In various further embodiments, the method further comprises administering an anti-TL1A therapy to the subject if the subject is diagnosed with the disease. In one embodiment, the disease is an IBD subtype, for example, an IBD subtype responsive to an anti-TL1 A therapy.

Various embodiments of the present invention provide a method for diagnosing susceptibility to an IBD subtype in a subject. The method may comprise or may consist essentially of or may consist of: obtaining a sample from the subject; assaying the expression level of one or more genes in the sample; comparing the expression level to a reference value of expression level of the one or more genes; and diagnosing susceptibility to the IBD subtype in the subject if the subject has an expression level different from the reference value, or not diagnosing susceptibility to the IBD subtype in the subject if the subject does not have an expression level different from the reference value.

In various methods described herein, the one or more biomarkers or genes assayed may be those described in Table 1, Table 4, Table 5 and/or Table 6 herein. In various methods described herein, the TL1A-associated disease may include but are not limited to fibrosis, ulcerative colitis (UC), Crohn's disease (CD), inflammatory bowel disease (IBD), chronic obstructive pulmonary disease, allergic lung inflammation, asthma, atherosclerosis, lupus, rheumatoid arthritis (RA), multiple sclerosis (MS), psoriasis, type 1 diabetes, lung carcinoma, colon carcinoma, leukemia, lymphoma, transplant rejection, graft versus host disease, or central nervous system injury. In various methods described herein, the IBD subtype may be characterized by being treatable with an anti-TL1A therapy, that is, an IBD subtype responsive to an anti-TL1A therapy.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts genes as markers for activation by TL1A in accordance with various embodiments of the present invention. The same data of quantitative PCR results are shown on different X-axis scales in the top panel and the bottom panel. Bar graphs represent how many folds these biomarkers increase their expression levels over the untreated control (UT group), either after the cells are primed with IL12 and IL18 (Primed group), or after the cells are stimulated TL1A together with IL12 and IL18 (Stimulated group). For example, IFNG expression level increase about 24 folds over the untreated control after being primed with IL12 and IL18, and increase about 283 folds over the untreated control after being primed with IL12 and IL18 and further stimulated with TL1A. “% ACTB” means that the expression level of a biomarker is a value standardized to β-Actin (ACTB) expression level, which serves as an internal standard. The Primed value and the Stimulated value are divided by the UT value to obtain the increase fold of each gene.

FIG. 2 depicts, in accordance with various embodiments of the present invention, effect of TL1A on IFNG mRNA. TL1A enhances IFN-γ Expression in CD4⁺ T Cells.

FIG. 3 depicts, in accordance with various embodiments of the present invention, intracellular IFN-γ in CD4⁺ PBL. Only a small population (1.5-3%) of CD4⁺ T cells upregulate IFN-γ production in response to TL1A. 1.5% of cell express IFN-γ in response to TL1A vs 8.5% in response to PMA/Ionomycin.

FIG. 4 depicts experimental designs in accordance with various embodiments of the present invention. The strategy shown in the left panel looks for genes differentially regulated in response to IL12+18 as compared to IL12+18+TL1A. The strategy shown in the right panel looks for genes differentially regulated in response to TL1A between IFN-γ positive and IFN-γ negative cell populations.

FIG. 5 depicts, in accordance with various embodiments of the present invention, capture of IFN-γ secreting cell population. Bivalent antibody binds to CD45 receptor on T cells and then capture IFN-γ protein. Protein is then detected by PE-anti-IFN-γ antibody.

FIG. 6 depicts, in accordance with various embodiments of the present invention, CD4⁺ IFN-sorted populations for new RNA-seq.

FIG. 7 depicts, in accordance with various embodiments of the present invention, IBD samples are not activated by TL1A (% ACTB).

FIG. 8 depicts, in accordance with various embodiments of the present invention, IBD samples are not activated by TL1A (% ACTB).

FIG. 9 depicts, in accordance with various embodiments of the present invention, genes activated by IL12+18 (% ACTB).

FIG. 10 depicts, in accordance with various embodiments of the present invention, genes activated by IL12+18 (% ACTB).

FIG. 11 depicts, in accordance with various embodiments of the present invention, differential gene expression in UT IBD samples.

FIG. 12 depicts, in accordance with various embodiments of the present invention, expression levels higher in IBD vs NL IL12+18-treated.

FIG. 13 depicts, in accordance with various embodiments of the present invention, expression levels lower in IBD vs NL TL1A-treated.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N. Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described.

“Subject” or “individual” or “patient” or “animal” or “mammal” refers to any subject, particularly a mammalian subject, for whom diagnosis, prognosis, treatment or therapy is desired. Mammalian subjects include, but are not limited to, humans; domestic animals; farm animals; zoo animals; sport animals; pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject. The term does not denote a particular age or sex. Thus, adult and newborn subjects, as well as fetuses, whether male or female, are intended to be included within the scope of this term.

“Biological sample” or “Sample” as used herein means any biological material from which nucleic acids and/or proteins can be obtained. As non-limiting examples, the term encompasses whole blood, plasma, saliva, cheek swab, or other bodily fluid or tissue that contains nucleic acids and/or proteins.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. In some embodiments, the disease condition is a TL1A-associated disease.

“Patient outcome” refers to whether a patient's health improves or worsens as a result of treatment as well as whether a patient survives or dies as a result of treatment. As provided in this invention, prescribing and administering an appropriate treatment (e.g., an anti-TL1A therapy or not) according to the specific conditions of individual patients increases their chances of health improvement and/or survival.

“TL1A” as used herein is a TNF-like cytokine factor encoded by the gene TNFSF15. Examples of TL1A include mouse TL1A such as NCBI reference sequence NM_177371.3, rat TL1A such as NCBI reference sequence AF520787.1, and human TL1A such as NCBI reference sequence NM_005118, NM_001204344.1, among others.

“Anti-TL1A therapy”, as used herein refers to therapeutic agents and methods that suppress TL1A gene expression, DR3 gene expression, or block the signaling of TL1A and DR3 (the receptor for TL1A) proteins. Examples of anti-TL1A therapy include, but are not limited to, an agent that specifically binds TL1A or DR3 and blocks TL1A-DR3 interaction, an anti-TL1A antibody blocking TL1A-DR3 signaling, an anti-DR3 antibody blocking TL1A-DR3 signaling, a soluble decoy DR3 polypeptide (e.g., a soluble DR3-Fc fusion protein), or a nucleic acid antagonist of TL1A or DR3, such as a ribozyme, aptamer or antisense molecule targeting TL1A or DR3, or a combination thereof.

As disclosed herein, the inventors discovered a TL1A-specific biomarker signature of 22 genes. In accordance with various embodiments herein, the invention includes devices, systems, and methods for stratifying patient population based on this biomarker signature to identify individuals most likely to been exposed in vivo to the pro-inflammatory effects of TL1A activation. As this particular population of patients would potentially benefit from an anti-TL1A therapy, for example, an anti-TL1A therapy may be prescribed or administrated to them. In another embodiment, by evaluating changes in the biomarker signature, one can monitor the progress and/or assess the effectiveness of an anti-TL1A therapy in a patient.

### Methods of Diagnosis and Treatments

In one embodiment, the invention provides for a method of selecting a treatment for a subject. In one embodiment, the present invention provides a method of diagnosis and/or treatment by obtaining a sample from the subject, assaying the expression level of one or more biomarkers associated with TL1A signaling in the sample, comparing the expression level to a reference value of expression level, and prescribing an anti-TL1A therapy to the subject if the subject has a high expression level relative to the reference value, or prescribing no anti-TL1A therapy to the subject if the subject does not have a high expression level relative to the reference value. In some embodiments, the method further comprises stimulating the sample with IL12, IL18, or TL1A, or a combination thereof, before assaying the expression level of one or more biomarkers associated with TL1A signaling in the sample. In another embodiment, the one or more biomarkers associated with TL1A signaling is listed in Table 1, Table 4, Table 5 and/or Table 6 herein.

In one embodiment, the invention provides for a method of identifying a subject likely to be responsive to an anti-TL1A therapy. In one embodiment, the present invention provides a method of identifying a subject likely to be responsive to an anti-TL1A therapy by obtaining a sample from the subject, assaying the expression level of one or more biomarkers associated with TL1A signaling in the sample, comparing the expression level to a reference value of expression level, and identifying the subject as likely to be responsive to an anti-TL1A therapy if the subject has a high expression level relative to the reference value, or identifying the subject as unlikely to be responsive to an anti-TL1A therapy if the subject does not have a high expression level relative to the reference value. In some embodiments, the method further comprises stimulating the sample with IL12, IL18, or TL1A, or a combination thereof, before assaying the expression level of one or more biomarkers associated with TL1A signaling in the sample. In another embodiment, the one or more biomarkers associated with TL1A signaling is listed in Table 1, Table 4, Table 5 and/or Table 6 herein.

In another embodiment, the invention provides for a method of treating a subject with an anti-TL1A therapy. In one embodiment, the method includes obtaining a sample from the subject, assaying the expression level of one or more biomarkers associated with TL1A signaling in the sample, comparing the expression level to a reference value of expression level, and administering an anti-TL1A therapy to the subject if the subject has a high expression level relative to the reference value, or administering no anti-TL1A therapy to the subject if the subject does not have a high expression level relative to the reference value. In some embodiments, the method further comprises stimulating the sample with IL12, IL18, or TL1A, or a combination thereof, before assaying the expression level of one or more biomarkers associated with TL1A signaling in the sample. In another embodiment, the one or more biomarkers are described in Table 1, Table 4, Table 5 and/or Table 6 herein.

In another embodiment, the invention provides for a method of diagnosing a TL1A-associated disease in a subject. In another embodiment, the method comprises obtaining a sample from the subject, assaying the expression level of one or more biomarkers associated with TL1A signaling in the sample, comparing the expression level to a reference value of expression level, and diagnosing a TL1A-associated disease in the subject if the subject has a high expression level relative to the reference value, or diagnosing no TL1A-associated disease in the subject if the subject does not have a high expression level relative to the reference value. In another embodiment, the one or more biomarkers are described in Table 1, Table 4, Table 5 and/or Table 6 herein.

In various embodiments, the invention provides for a method diagnosing susceptibility to a TL1A-associated disease in a subject. In one embodiment, the method comprises: obtaining a sample from the subject, assaying the expression level of one or more biomarkers associated with TL1A signaling in the sample, comparing the expression level to a reference value of expression level, and diagnosing susceptibility to a TL1A-associated disease in the subject if the subject has a high expression level relative to the reference value, or diagnosing no susceptibility to a TL1A-associated disease in the subject if the subject does not have a high expression level relative to the reference value. In another embodiment, the one or more biomarkers associated with TL1A signaling is described in Table 1, Table 4, Table 5 and/or Table 6 herein. In another embodiment, the TL1A-associated disease includes fibrosis. In another embodiment, the TL1A-associated disease includes Inflammatory Bowel Disease (IBD).

In various embodiments, the invention provides a method of treating a disease in a subject. In one embodiment, the method comprises: administering an anti-TL1A therapy to the subject, thereby treating the disease, wherein the subject has a high expression level relative to a reference value of one or more biomarkers associated with TL1A signaling. In another embodiment, the one or more biomarkers are described in Table 1, Table 4, Table 5 and/or Table 6 herein.

In various embodiments, the invention provides for a method of diagnosing an IBD subtype in a subject. In one embodiment, the method comprises: obtaining a sample from the subject; assaying the expression level of one or more biomarkers associated with TL1A signaling in the sample; comparing the expression level to a reference value of expression level; and diagnosing the IBD subtype in the subject if the subject has a high expression level relative to the reference value, or not diagnosing the IBD subtype in the subject if the subject does not have a high expression level relative to the reference value. In some embodiments, the method further comprises stimulating the sample with IL12, IL18, or TL1A, or a combination thereof, before assaying the expression level of one or more biomarkers associated with TL1A signaling in the sample. In another embodiment, the one or more biomarkers are described in Table 1, Table 4, Table 5 and/or Table 6 herein. In one embodiment, the IBD subtype is characterized by being treatable with an anti-TL1A therapy.

In various embodiments, the invention provides for a method for diagnosing a disease in a subject. In one embodiment, the method comprises: obtaining a sample from a subject; assaying the expression level of one or more genes listed in Table 1, Table 4, Table 5 and/or Table 6 herein in the sample; comparing the expression level to a reference value of expression level of the one or more genes; and diagnosing a disease in the subject according to the relative difference between the expression level and the reference value. In some embodiments, the method further comprises stimulating the sample with IL12, IL18, or TL1A, or a combination thereof, before assaying the expression level of one or more genes in the sample. In some embodiments, the method further comprises diagnosing the disease in the subject if the subject has an expression level higher than the reference value, or not diagnosing the disease in the subject if the subject does not have an expression level higher than the reference value. In other embodiments, the method further comprises diagnosing the disease in the subject if the subject to has an expression level lower than the reference value, or not diagnosing the disease in the subject if the subject does not have an expression level lower than the reference value. In various further embodiments, the method further comprises prescribing an anti-TL1A therapy to the subject if the subject is diagnosed with the disease. In various further embodiments, the method further comprises administering an anti-TL1A therapy to the subject if the subject is diagnosed with the disease.

In various embodiments, the disease is a TL1A-associated disease. In various embodiments, the disease is fibrosis, Crohn's disease (CD), inflammatory bowel disease (IBD), chronic obstructive pulmonary disease, allergic lung inflammation, asthma, atherosclerosis, lupus, rheumatoid arthritis (RA), multiple sclerosis (MS), psoriasis, type 1 diabetes, lung carcinoma, colon carcinoma, leukemia, lymphoma, transplant rejection, graft versus host disease, or central nervous system injury. In various embodiments, the disease is a subtype of a disease, such as an IBD subtype responsive to an anti-TL1A therapy.

In various embodiments, the method comprises diagnosing the disease in the subject if the subject has an expression profile different from a reference profile, or not diagnosing the disease in the subject if the subject does not have an expression profile different from a reference profile. In accordance with the present invention, the expression profile may comprise a plurality of gene expression levels, in which some gene expression levels may be higher and other gene expression levels may be lower than the reference profile.

In various embodiments, the invention provides for a method of diagnosing susceptibility to an IBD subtype in a subject. In one embodiment, the method comprises: obtaining a sample from the subject; assaying the expression level of one or more biomarkers associated with TL1A signaling in the sample; comparing the expression level to a reference value of expression level; and diagnosing susceptibility to the IBD subtype in the subject if the subject has a high expression level relative to the reference value, or diagnosing no susceptibility to the IBD subtype in the subject if the subject does not have a high expression level relative to the reference value. In another embodiment, the one or more biomarkers are described in Table 1, Table 4, Table 5 and/or Table 6 herein. In one embodiment, the IBD subtype is characterized by being treatable with an anti-TL1A therapy.

In various embodiments, the invention provides for a method for diagnosing susceptibility to an IBD subtype in a subject. In one embodiment, the method comprises: obtaining a sample from the subject; assaying the expression level of one or more genes in the sample; comparing the expression level to a reference value of expression level of the one or more genes; and diagnosing susceptibility to the IBD subtype in the subject if the subject has an expression level different from the reference value, or not diagnosing susceptibility to the IBD subtype in the subject if the subject does not have an expression level different from the reference value. In various embodiments, the one or more genes is listed in Table 1, Table 4, Table 5 and/or Table 6 herein. In various embodiments, the IBD subtype is a subtype responsive to an anti-TL1A therapy.

In some embodiments, assaying the expression level of one or more genes listed in Table 1, Table 4, Table 5 and/or Table 6 herein in the sample comprise assaying at least two, three, four, or five genes listed in Table 1, Table 4, Table 5 and/or Table 6 herein. In other embodiments, assaying the expression level of one or more genes listed in Table 1, Table 4, Table 5 and/or Table 6 herein in the sample comprise assaying all of genes listed in Table 1, Table 4, Table 5 and/or Table 6 herein. In another embodiment, assaying the expression level of one or more genes listed in Table 1, Table 4, Table 5 and/or Table 6 herein in the sample comprise assaying any number (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55) of genes listed in Table 1, Table 4, Table 5 and/or Table 6 herein. In various embodiments, the methods described herein comprise assaying the expression level of one or more genes listed Table 6.

In various embodiments, the methods described herein comprise assaying the expression level of one or more genes selected from the group consisting of BIRC3, C17orf49, CCL20, CSF2, CD274, CD74, EPSTI1, FAS, GBP1, GBP4, GBP5, HAPLN3, IFNG, IRF1, NFKBIA, NFKB2, RELB, RGS1, SGK1, STAT1, TAP1, and TRAFD1. In various embodiments, the methods described herein comprise assaying the expression level of one or more genes selected from the group consisting of BATF, CCL20, CD274, CD83, CDKN1A, CHAC1, CSF2, DUSP5, FEZ1, GADD45G, HMSD, IFNG, IL22, IL26, IL411, IRF8, LTA, MFSD2A, MYO1B, NFKBIA, RPL21, SGK1, TNFRSF18, TNFRSF4, TRAF4, and XIST.

### Subject

In accordance with various embodiments herein, the subject can be human, monkey, ape, dog, cat, cow, horse, goat, pig, rabbit, mouse or rat. In one embodiment, the subject has a symptom of a TL1A-associated disease, is suspected of having a TL1A-associated disease, or is diagnosed with a TL1A-associated disease. In another embodiment, the subject has received, is receiving, or will receive an anti-TL1A therapy. In another embodiment, the subject has been, is being, or will be treated for a TL1A-associated disease. In another embodiment, the subject is in complete or partial remission, or has a recurrence of a TL1A-associated disease. In one embodiment, the subject has a symptom of an IBD subtype. In another embodiment, the subject is suspected of having an IBD subtype. In some embodiments, the IBD subtype is a subtype responsive to an anti-TL1A therapy.

### Sample

In one embodiment, the sample comprises a T cell, CD4⁺ T cell, CD8⁺ T cell, CD56⁺ T cell, CD45R0⁺ T cell, CD45RA⁺ T cell, NK cell, peripheral blood mononuclear cell (PBMC), or peripheral blood lymphocyte (PBL), or a combination thereof. In various embodiments, the sample is a cell, tissue, or body fluid. In various embodiments, the sample can be serum, urine, blood, plasma, saliva, semen, lymph, or a combination thereof. In various embodiments, the sample can be obtained before, during, or after a treatment of TL1A-associated disease. In various embodiments, the sample can be obtained before, during, or after an anti-TL1A therapy.

### TL1A-Associated Disease

In accordance with various embodiments herein, the TL1A-associated disease is fibrosis, Crohn's disease (CD), inflammatory bowel disease (IBD), chronic obstructive pulmonary disease, allergic lung inflammation, asthma, atherosclerosis, lupus, rheumatoid arthritis (RA), multiple sclerosis (MS), psoriasis, type 1 diabetes, lung carcinoma, colon carcinoma, leukemia, lymphoma, transplant rejection, graft versus host disease, or central nervous system injury.

Examples of “TL1A-associated diseases” include, but are not limited to, fibrosis, Crohn's disease (CD), inflammatory bowel disease (IBD), chronic obstructive pulmonary disease, allergic lung inflammation, asthma, atherosclerosis, lupus, rheumatoid arthritis (RA), multiple sclerosis (MS), psoriasis, type 1 diabetes, lung carcinoma, colon carcinoma, leukemia, lymphoma, transplant rejection, graft versus host disease, or central nervous system injury.

IBD includes several forms of inflammatory diseases and conditions affecting various parts of the gastrointestinal (GI) tract, such as the colon and small intestine. Examples of IBD include, but are not limited to, Crohn's disease (CD), ulcerative colitis (UC), other forms of colitis such as collagenous colitis, lymphocytic colitis, ischaemic colitis, diversion colitis, Behçet's disease, and indeterminate colitis, among others. Crohn's disease (CD) and ulcerative colitis (UC) are two main forms of IBD. Hallmarks of IBD can include inflammation of the digestive tract in compartments of the epithelial mucosa or transmural lesions in the bowel wall.

### Expression Level Assay—RNA

In various embodiments, assaying the expression level of one or more biomarkers associated with TL1A signaling in the sample comprises assaying an mRNA level. In various embodiments, assaying an mRNA level comprises using RNA sequencing, northern blot, in situ hybridization, hybridization array, serial analysis of gene expression (SAGE), reverse transcription PCR, real-time PCR, real-time reverse transcription PCR, or quantitative PCR, or a combination thereof.

In various embodiments, assaying an mRNA level comprises contacting the sample with a polynucleotide probe capable of specifically hybridizing to mRNA of one or more biomarkers associated with TL1A signaling and thereby forming a probe-target hybridization complex.

Hybridization-based RNA assays include, but are not limited to, traditional “direct probe” methods such as, northern blot or in situ hybridization (e.g., Angerer (1987) Meth. Enzymol 152: 649). The methods can be used in a wide variety of formats including, but not limited to, substrate (e.g. membrane or glass) bound methods or array-based approaches. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. Preferred probes are sufficiently long so as to specifically hybridize with the target nucleic acid(s) under stringent conditions. The preferred size range is from about 200 bases to about 1000 bases. Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), Pinkel, et al. (1998) Nature Genetics 20: 207-211, and/or Kallioniemi (1992) Proc. Natl Acad Sci USA 89:5321-5325 (1992). In some applications, it is necessary to block the hybridization capacity of repetitive sequences. Thus, in some embodiments, tRNA, human genomic DNA, or Cot-I DNA is used to block non-specific hybridization.

In various embodiments, assaying an mRNA level comprises contacting the sample with one or more polynucleotide primers capable of specifically hybridizing to mRNAs of genes listed in Table 1, Table 4, Table 5 and/or Table 6, forming a primer-template hybridization complex, and performing a PCR reaction. In some embodiments, the one or more polynucleotide primers are primers listed in Table 2. In other embodiments, the one or more polynucleotide primers comprises about 15-45, 20-40, or 25-35 bp sequences that are identical (for forward primers) or complementary (for reverse primers) to sequences of genes listed in Table 1, Table 4, Table 5 and/or Table 6. As a non-liming example, the one or more polynucleotide primers for INFG (e.g., NM_000619.2 with 1240 bp) can comprise sequences that are identical (for forward primers) or complementary (for reverse primers) to INFG's bp 1-20, 5-25, 10-30, 15-35, 20-40, 25-45, 30-50, so on and so forth, until the end of INFG, 1201-1220, 1205-25, 1210-1230, 1215-1235, 1220-1240. While not listed here exhaustively because of the space, all these polynucleotide primers for INFG and other genes listed in Table 1, Table 4, Table 5 and/or Table 6 can be used in the present invention. In various embodiments, the one or more polynucleotide primers are labeled with radioisotopes or fluorescent molecules. As the labeled primers emit radio or fluorescent signals, the PCR products containing the labeled primers can be detected and analyzed with a variety of imaging equipment.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR anlaysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and sybr green. Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Expression Level Assay—Protein

In various embodiments, assaying the expression level of one or more biomarkers associated with TL1A signaling in the sample comprises assaying a protein level. In various embodiments, assaying a protein level comprises using western blot, enzyme-linked immunosorbent assay (ELISA), radioimmunoassay, or mass spectrometry, or a combination thereof.

In various embodiments, assaying a protein level comprises contacting the sample with antibodies capable of specifically binding to proteins of genes listed in Table 1, Table 4, Table 5 and/or Table 6 and thereby forming antigen-antibody complexes. In the methods and assays of the invention, the expression levels of proteins encoded by biomarker genes listed in Table 1, Table 4, Table 5 and/or Table 6, or fragments or variants thereof can be determined using antibodies specific for those individual proteins or fragments or variants thereof and detecting immunospecific binding of each antibody to its respective cognate biomarker protein.

Antibodies, both polyclonal and monoclonal, can be produced by a skilled artisan either by themselves using well known methods or they can be manufactured by service providers who specialize making antibodies based on known protein sequences. In the present invention, the protein sequences of biomarker gens are known and thus production of antibodies against them is a matter of routine.

For example, production of monoclonal antibodies can be performed using the traditional hybridoma method by first immunizing mice with an antigen which may be an isolated protein of choice or fragment thereof (for example, a protein encode by a biomarker gene listed in Table 1, Table 4, Table 5 and/or Table 6, or a fragment thereof or a variant thereof) and making hybridoma cell lines that each produce a specific monoclonal antibody. The antibodies secreted by the different clones are then assayed for their ability to bind to the antigen using, e.g., ELISA or Antigen Microarray Assay, or immuno-dot blot techniques. The antibodies that are most specific for the detection of the protein of interest can be selected using routine methods and using the antigen used for immunization and other antigens as controls. The antibody that most specifically detects the desired antigen and protein and no other antigens or proteins are selected for the processes, assays and methods described herein. The best clones can then be grown indefinitely in a suitable cell culture medium. They can also be injected into mice (in the peritoneal cavity, surrounding the gut) where they produce an antibody-rich ascites fluid from which the antibodies can be isolated and purified. The antibodies can be purified using techniques that are well known to one of ordinary skill in the art.

Any suitable immunoassay method may be utilized, including those which are commercially available, to determine the expression level of a biomarker protein or a variant thereof assayed according to the invention. Extensive discussion of the known immunoassay techniques is not required here since these are known to those of skill in the art. Typical suitable immunoassay techniques include sandwich enzyme-linked immunoassays (ELISA), radioimmunoassays (RIA), competitive binding assays, homogeneous assays, heterogeneous assays, etc.

For example, in the assays of the invention, “sandwich-type” assay formats can be used. An alternative technique is the “competitive-type” assay. In a competitive assay, the labeled probe is generally conjugated with a molecule that is identical to, or an analog of, the analyte. Thus, the labeled probe competes with the analyte of interest for the available receptive material. Competitive assays are typically used for detection of analytes such as haptens, each hapten being monovalent and capable of binding only one antibody molecule.

The antibodies can be labeled. In some embodiments, the detection antibody is labeled by covalently linking to an enzyme, label with a fluorescent compound or metal, label with a chemiluminescent compound. For example, the detection antibody can be labeled with catalase and the conversion uses a colorimetric substrate composition comprises potassium iodide, hydrogen peroxide and sodium thiosulphate; the enzyme can be alcohol dehydrogenase and the conversion uses a colorimetric substrate composition comprises an alcohol, a pH indicator and a pH buffer, wherein the pH indicator is neutral red and the pH buffer is glycine-sodium hydroxide; the enzyme can also be hypoxanthine oxidase and the conversion uses a colorimetric substrate composition comprises xanthine, a tetrazolium salt and 4,5-dihydroxy-1,3-benzene disulphonic acid. In one embodiment, the detection antibody is labeled by covalently linking to an enzyme, label with a fluorescent compound or metal, or label with a chemiluminescent compound.

Direct and indirect labels can be used in immunoassays. A direct label can be defined as an entity, which in its natural state, is visible either to the naked eye or with the aid of an optical filter and/or applied stimulation, e.g., ultraviolet light, to promote fluorescence. Examples of colored labels which can be used include metallic sol particles, gold sol particles, dye sol particles, dyed latex particles or dyes encapsulated in liposomes. Other direct labels include radionuclides and fluorescent or luminescent moieties. Indirect labels such as enzymes can also be used according to the invention. Various enzymes are known for use as labels such as, for example, alkaline phosphatase, horseradish peroxidase, lysozyme, glucose-6-phosphate dehydrogenase, lactate dehydrogenase and urease.

The antibody can be attached to a surface. Examples of useful surfaces on which the antibody can be attached for the purposes of detecting the desired antigen include nitrocellulose, PVDF, polystyrene, and nylon.

In some embodiments of the processes, assays and methods described herein, detecting the level of antibodies reactive to a biomarker protein or a variant thereof includes contacting the sample from the cancer patient with an antibody or a fragment thereof that specifically binds a biomarker protein or a variant thereof, forming an antibody-protein complex between the antibody and the biomarker protein or a variant thereof present in the sample, washing the sample to remove the unbound antibody, adding a detection antibody that is labeled and is reactive to the antibody bound to the biomarker protein or a variant thereof in the sample, washing to remove the unbound labeled detection antibody and converting the label to a detectable signal, wherein the detectable signal is indicative of the level of biomarker protein or a variant thereof in the sample from the patient. In some embodiments, the effector component is a detectable moiety selected from the group consisting of a fluorescent label, a radioactive compound, an enzyme, a substrate, an epitope tag, electron-dense reagent, biotin, digonigenin, hapten and a combination thereof. In some embodiments, the detection antibody is labeled by covalently linking to an enzyme, labeled with a fluorescent compound or metal, labeled with a chemiluminescent compound. The level of biomarker protein may be obtained by assaying a light scattering intensity resulting from the formation of an antibody-protein complex formed by a reaction of biomarker protein in the sample with the antibody, wherein the light scattering intensity of at least 10% above a control light scattering intensity indicates the likelihood of chemotherapy resistance.

### Reference Value of Expression Level

In various embodiments, the reference value of expression level is the median or mean expression level from a population of subjects who have no TL1A-associated disease. In one embodiment, the reference value of expression level is the median or mean expression level from a population of subjects who have no IBD. In various embodiments, the reference value of expression level is the median or mean expression level from a population of subjects who are unlikely to be responsive to an anti-TL1A therapy. In various embodiments, the reference value of expression level is the median or mean expression level from a population of subjects who are not responsive to an anti-TL1A therapy. In additional embodiments, the reference value is the expression level of a biomarker gene or a variant thereof in a sample obtained from the subject at a different (for example, an earlier) time point, such as during diagnosis, before treatment, during treatment, after treatment or a combination thereof.

Various statistical methods, for example, a two-tailed student t-test with unequal variation, may be used to measure the differences in expression levels of a biomarker gene between the subject's sample and a control sample from a normal/healthy individual, or a reference value of expression level generate by computer algorithm pooling many control samples, as described herein. A significant difference may be achieved where the p value is equal to or less than 0.05.

In various embodiments, the expression level of a biomarker gene or a variant thereof in the subject as compared to the reference value is higher by at least or about 5, 10, 15, 20, 25, 30, 35, 40, 45, 50, 55, 60, 65, 70, 75, 80, 85, 90, 95, or 100%. In various embodiments, the expression level of a biomarker gene or a variant thereof in the subject as compared to the reference value is increased by at least or about 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, 2.5-fold, 2.6-fold, 2.7-fold, 2.8-fold, 2.9-fold, 3-fold, 4-fold, 5-fold, 6-fold, 7-fold, 8-fold, 9-fold or 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold.

### Anti-TL1A Therapy

In various embodiments, the anti-TL1A therapy comprises an agent that specifically binds TL1A or DR3 and blocks TL1A-DR3 interaction. In one embodiment, the anti-TL1A therapy comprises an anti-TL1A antibody or a fragment thereof, antagonistic anti-TL1A antibodies, or an isolated antigen-binding polypeptide that binds specifically to TL1A, or a combination thereof. In one embodiment, the anti-TL1A therapy comprises a soluble form of TL1A that specifically binds DR3. In one embodiment, the anti-TL1A therapy comprises an anti-DR3 antibody or a fragment thereof, antagonistic anti-DR3 antibodies, an isolated antigen-binding polypeptide that binds specifically to DR3, or a combination thereof. In one embodiment, the anti-TL1A therapy comprises a soluble form of DR3 that specifically binds TL1A. In one embodiment, the anti-TL1A therapy comprises a soluble decoy DR3 polypeptide, a polypeptide comprising a DR3 extracellular domain, a DR3-Fc protein, or a polypeptide comprising a DR3 pre-ligand assembly domain (a DR3-PLAD peptide), or a combination thereof. In one embodiment, the anti-TL1A therapy comprises a dominant negative DR3. In one embodiment, the anti-TL1A therapy comprises agents targeting TL1A or DR3 expression (e.g., ribozymes, aptamers and antisense nucleic acids), a nucleic acid antagonist of TL1A, or a nucleic acid antagonist of DR3), or a combination thereof. In one embodiment, the anti-TL1A therapy comprises GEP and GEP peptides including the peptide(s) denoted Atsttrin, Atsttrin-α variants, or a combination thereof.

The duration and/or dose of treatment with anti-TL1A therapies may vary according to the particular anti-cancer agent or combination thereof. An appropriate treatment time for a particular anti-TL1A therapeutic agent will be appreciated by the skilled artisan. The invention contemplates the continued assessment of optimal treatment schedules for each anti-TL1A therapeutic agent, where the TL1A-specific biomarker signature of the subject as determined by the methods of the invention is a factor in determining optimal treatment doses and schedules.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Biomarker Signature for TL1A Signaling

CD4⁺ T cells from normal individuals were treated with recombinant TL1A following priming with IL12 and IL18. RNA sequencing was utilized to measure TL1A mediated gene activation and to identify biomarkers responsive to TL1A signaling.

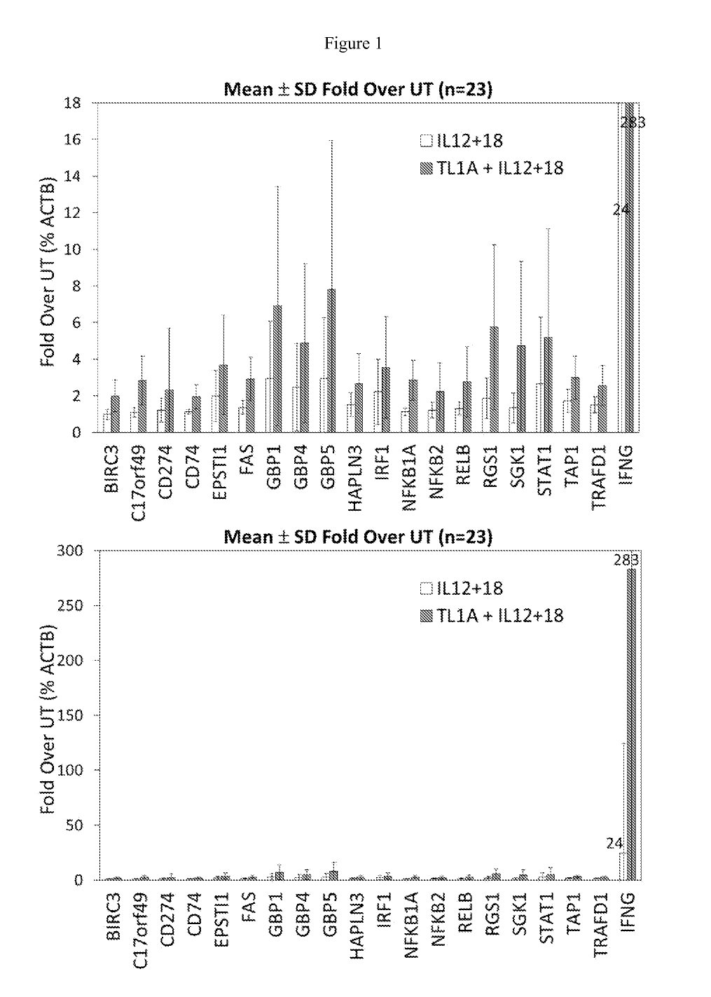

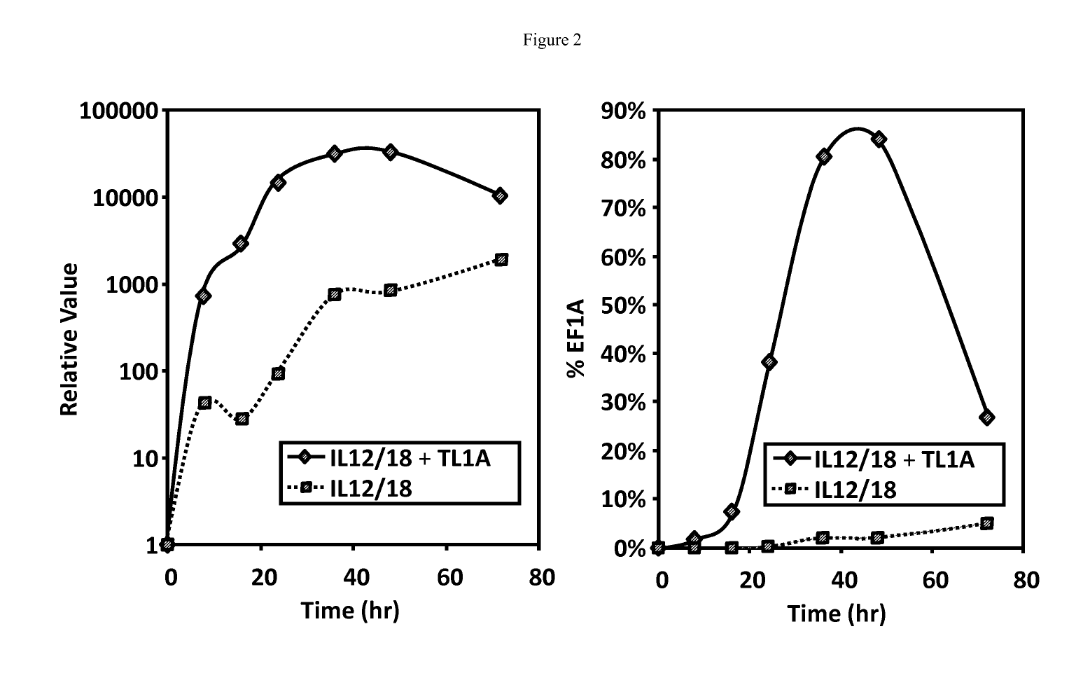

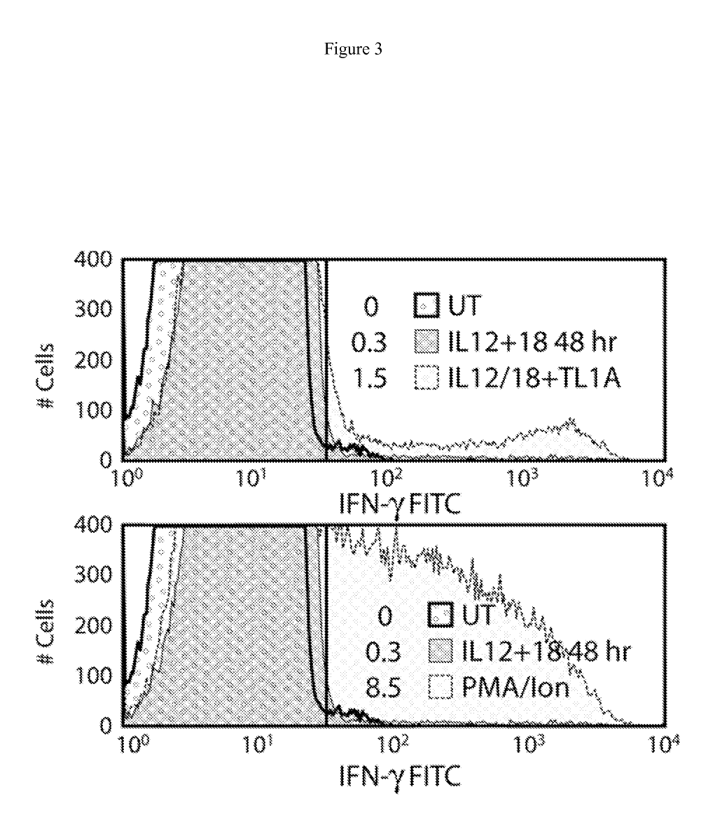

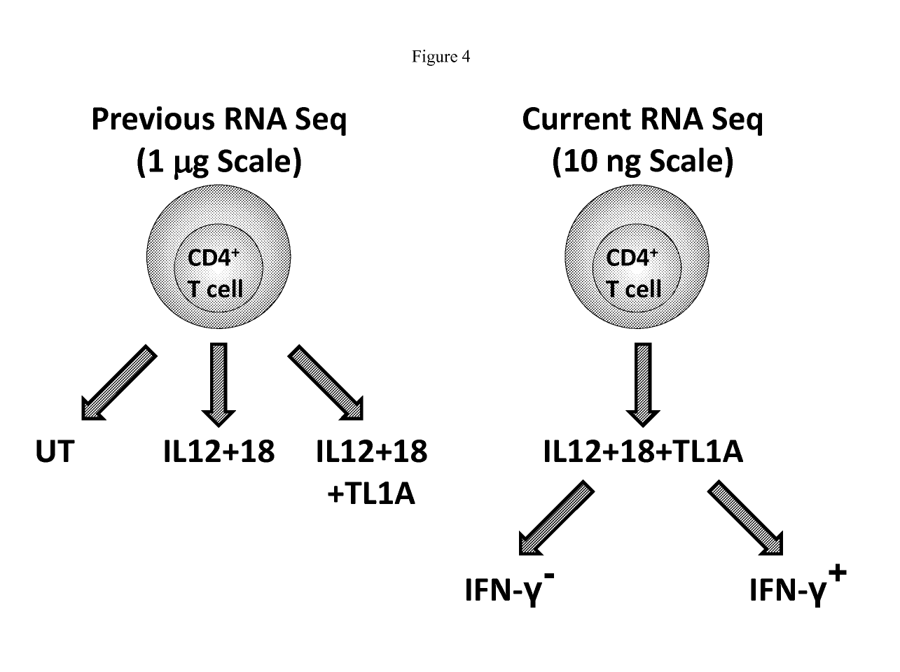

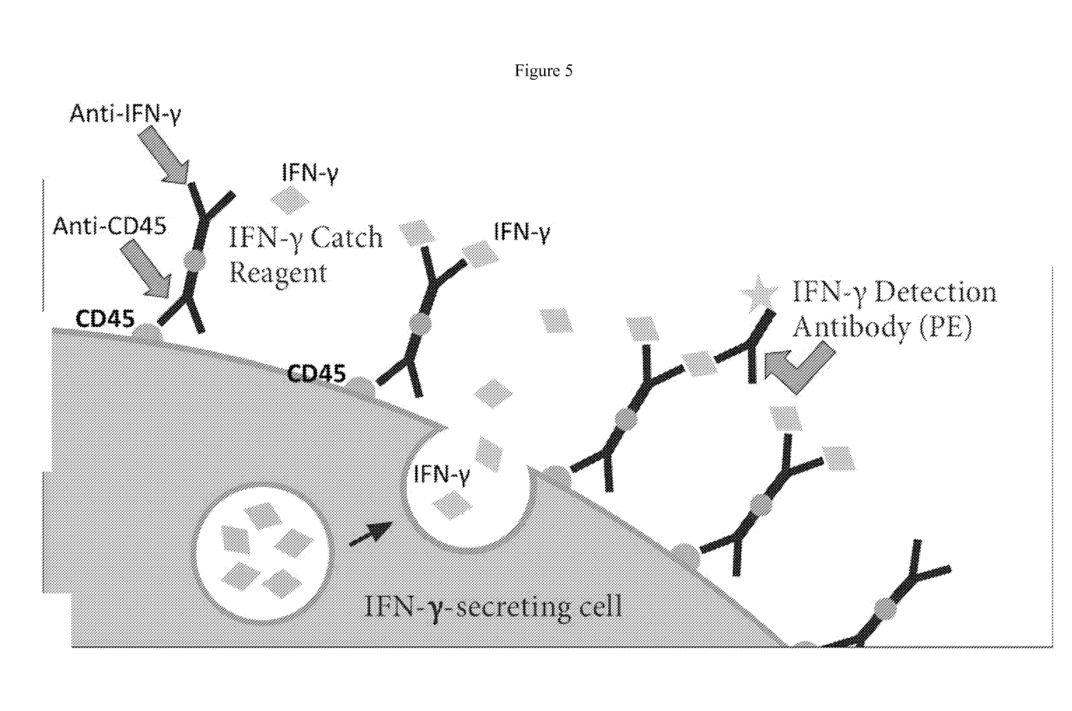

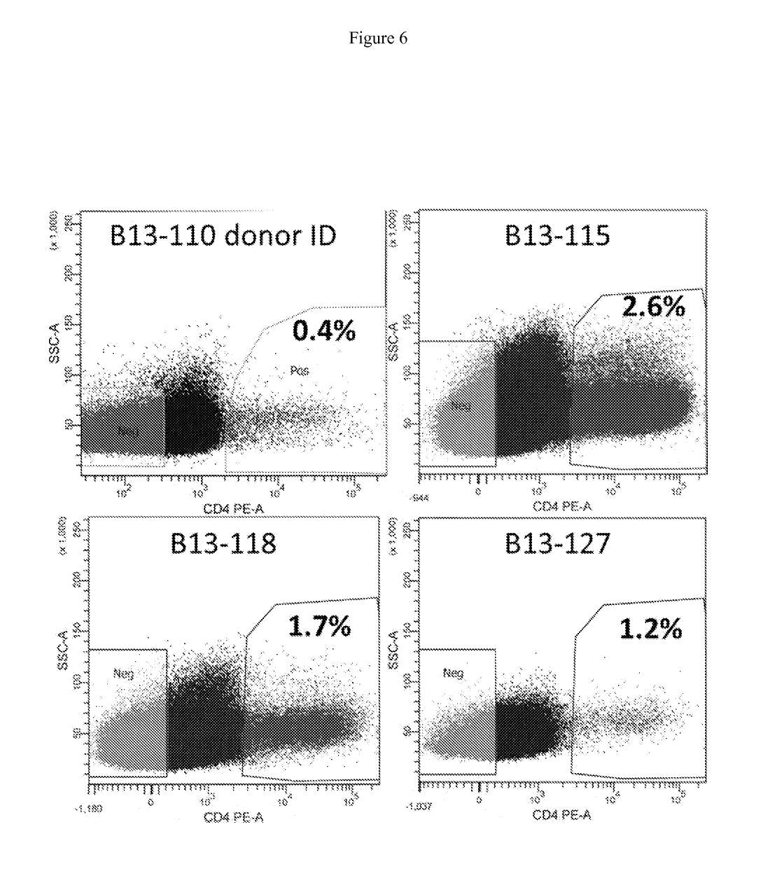

In one example, CD4⁺ cells were isolated from normal donors, rested overnight, and then treated for 8 hours in three groups: untreated (UT), primed (IL12+IL18), and stimulated (IL12+IL18+TL1A)). RNA were isolated from the cells and used for Fluidigm qPCR for 24 genes (22 biomarker genes and 2 housekeeping ActB and EEF1A1). Real-time PCR of 22 genes validated these genes as markers for activation by TL1A (FIG. 1). Genes used for validation are listed in Table 1.

TABLE 1
TL1A-specific Biomarker Signature

| Gene Name | Gene Name | Gene Name |
|---|---|---|
| BIRC3 | GBP1 | RGS1 |
| C17orf49 | GBP4 | SGK1 |
| CCL20 | GBP5 | STAT1 |
| CSF2 | HAPLN3 | TAP1 |
| CD274 | IRF1 | TRAFD1 |
| CD74 | NFKBIA | IFNG |
| EPSTI1 | NFKB2 | |
| FAS | RELB | |

### Example 2

All primers were optimized for efficiency and absence of off-target amplification using cyber-green qPCR.

TABLE 2
Sequences of Primers and Probes

| | | | Primer |
|---|---|---|---|
| Transcript | NCBI ID | Primer Sequence | SEQ ID NO: |
| IFNG | NM_000619.2 | TTGGGTTCTCTTGGCTGTTACT | SEQ ID NO: 1 |
| | | ATCCGCTACATCTGAATGACCTG | SEQ ID NO: 2 |
| RGS1 | NM_002922.3 | CTTGCCAACCAAACTGGTCAAA | SEQ ID NO: 3 |
| | | ACAAGCCAGCCAGAACTCAATA | SEQ ID NO: 4 |
| GBP5 | NM_001134486.1 | GGTTGGCGGCGATTCAAAG | SEQ ID NO: 5 |
| | | AGTCCTCTGGGCGTGCT | SEQ ID NO: 6 |
| GBP1 | NM_002053.2 | ACTTCAGGAACAGGAGCAACT | SEQ ID NO: 7 |
| | | GGTACATGCCTTTCGTCGTCT | SEQ ID NO: 8 |
| IRF1 | NM_002198.2 | AGGAGGTGAAAGACCAGAGC | SEQ ID NO: 9 |
| | | CTCTTAGCATCTCGGCTGGA | SEQ ID NO: 10 |
| STAT1 | NM_007315 | GTTTGACGAGGTGTCTCGGATAG | SEQ ID NO: 11 |
| | | AACTGTCGCCAGAGAAGATGAA | SEQ ID NO: 12 |
| C17orf49 | NM_001142798.1 | TGCTCTGAACGACTCCGATG | SEQ ID NO: 13 |
| | | AGAATCCAGGGTCAGGCTGT | SEQ ID NO: 14 |
| SGK1 | NM_001143676.1 | AGCAAGACACAAGGCAGAAGA | SEQ ID NO: 15 |
| | | CAGAACATTCCGCTCCGACATA | SEQ ID NO: 16 |
| GBP4 | NM_052941.4 | GGACAGAGCAATGGGTGAGAG | SEQ ID NO: 17 |
| | | ACTAGACAAATGGGGGCCATC | SEQ ID NO: 18 |
| EPSTI1 | NM_001002264.1 | GCAGCAGCAAGAGCAAGAAA | SEQ ID NO: 19 |
| | | GGAGTCGGTCCAGAAAAGCA | SEQ ID NO: 20 |
| CD274 | NM_014143.2 | GTTGAAGGACCAGCTCTCCC | SEQ ID NO: 21 |
| | | CTTGTAGTCGGCACCACCAT | SEQ ID NO: 22 |
| RELB | NM_006509.2 | ATTGACCCCTACAACGCTGG | SEQ ID NO: 23 |
| | | TCCGCAGCTCTGATGTGTTT | SEQ ID NO: 24 |
| NFKBIA | NM_020529.2 | AAGTGATCCGCCAGGTGAAG | SEQ ID NO: 25 |
| | | CTGCTCACAGGCAAGGTGTA | SEQ ID NO: 26 |
| NFKB2 | NM_001077494.2 | CGCTTCTCTGCCTTCCTTAGAG | SEQ ID NO: 27 |
| | | AGCCTGCTGTCTTGTCCATT | SEQ ID NO: 28 |
| CD74 | NM_001025159.1 | GCACTCCTTGGAGCAAAAGC | SEQ ID NO: 29 |
| | | AAGACACACCAGCAGTAGCC | SEQ ID NO: 30 |
| TRAFD1 | NM_001143906.1 | GTGGTAGGAGTGAAGGTGGC | SEQ ID NO: 31 |
| | | GCACAGAAGACAGGAACCGA | SEQ ID NO: 32 |
| TAP1 | NM_000593.5 | TTTGAGTACCTGGACCGCAC | SEQ ID NO: 33 |
| | | AATGTCAGCCCCTGTAGCAC | SEQ ID NO: 34 |
| BIRC3 | NM_182962 | AGTGGTTTCCAAGGTGTGAGT | SEQ ID NO: 35 |
| | | CTGGGCTGTCTGATGTGGATAG | SEQ ID NO: 36 |
| HAPLN3 | NM_178232 | CAACGGCTTCTACTACTCCAACA | SEQ ID NO: 37 |
| | | CACCACCAGCTTCACTCCATTA | SEQ ID NO: 38 |
| FAS | NM_000043 | ACTGTGACCCTTGCACCAAAT | SEQ ID NO: 39 |
| | | GCCACCCCAAGTTAGATCTGG | SEQ ID NO: 40 |
| EEF1A1 | NM_001402.5 | CACACGGCTCACATTGCAT | SEQ ID NO: 41 |
| | | CACGAACAGCAAAGCGACC | SEQ ID NO: 42 |
| ACTB | NM_001101.3 | CGTGCTGCTGACCGAGG | SEQ ID NO: 43 |
| | | AAGGTCTCAAACATGATCTGGGT | SEQ ID NO: 44 |
| CSF2 | NM_000758.3 | GAGACACTGCTGCTGAGATGAA | SEQ ID NO: 45 |
| | | GGCTCCTGGAGGTCAAACAT | SEQ ID NO: 46 |
| CCL20 | NM_004591.2 | GTCTGTGTGCGCAAATCCAA | SEQ ID NO: 47 |
| | | GAAACCTCCAACCCCAGCAA | SEQ ID NO: 48 |
| GADD45B-F | | TGA ATG TGG ACC CAG ACA GC | SEQ ID NO: 49 |
| GADD45B-R | | ACT GGA TGA GCG TGA AGT GG | SEQ ID NO: 50 |
| MFSD2A-F | | GGA GCA GAG AGA ACC CTA TGA | SEQ ID NO: 51 |
| | | A | |
| MFSD2A-R | | AGG TGA AGA GGA AGC CAG TAA | SEQ ID NO: 52 |
| TRAF4-F | | CGT GAT CTA CCT GCA CAC TTG | SEQ ID NO: 53 |
| TRAF4-R | | ACA TAC CCT CAT GGC TCT CAT A | SEQ ID NO: 54 |
| TNFRSF4-F | | TGT AAC CTC AGA AGT GGG AGT G | SEQ ID NO: 55 |
| TNFRSF4-R | | ACA GTC AAC TCC AGG CTT GT | SEQ ID NO: 56 |
| SLAMF7- | | CCA ACA TGC CTC ACC CTC AT | SEQ ID NO: 57 |
| sense | | | |
| SLAMF7- | | GGA ACC GAC CAG CTC TTT CA | SEQ ID NO: 58 |
| Antisense | | | |
| SLC7A5-F | | CTC TTC CTG ATC GCC GTC TC | SEQ ID NO: 59 |
| SLC7A5-R | | GAC CAC CTG CAT GAG CTT CT | SEQ ID NO: 60 |
| SOD2-F | | CAC TGC AAG GAA CAA CAG GC | SEQ ID NO: 61 |
| SOD2-R | | TGC TCC CAC ACA TCA ATC CC | SEQ ID NO: 62 |
| IRF4-F | | GCC CAG CAG GTT CAC AAC TA | SEQ ID NO: 63 |
| IRF4-R | | AGG TGG GGC ACA AGC ATA AA | SEQ ID NO: 64 |
| IL22-F | | TGG GGA GAA ACT GTT CCA CG | SEQ ID NO: 65 |
| IL22-R | | TGT GCT TAG CCT GTT GCT GA | SEQ ID NO: 66 |
| IL26-F | | AAC GAT TCC AGA AGA CCG CA | SEQ ID NO: 67 |
| IL26-R | | GAA AGT CCT CCA CAA AGC GT | SEQ ID NO: 68 |
| FEZ1-F | | GCT CAC AGC AGA TCA GGT AAT | SEQ ID NO: 69 |
| | | TG | |
| FEZ1-R | | TTT CTC CTC CAT CCT CTT CTT CC | SEQ ID NO: 70 |
| CHAC1-F | | GCA GGG AGA CAC CTT CCA TC | SEQ ID NO: 71 |
| CHAC1-R | | TCT TCA AGG AGC GTC ACC AC | SEQ ID NO: 72 |
| MYO1B-F | | ACC GTG CTA ATG GGA AGA GTA | SEQ ID NO: 73 |
| MYO1B-R | | CAC CAA TGG AAC CTC TGA CTT G | SEQ ID NO: 74 |
| HMSD- | | CAA CGG GCT CTT TGG AGA AA | SEQ ID NO: 75 |
| sense | | | |
| HMSD- | | CGT GTT GTG GAC TTC TCT GTA | SEQ ID NO: 76 |
| Antisense | | TC | |
| CD83-F | | CGA AGA TGT GGA CTT GCC CT | SEQ ID NO: 77 |
| CD83-R | | GGG GTG TCT CCA TCC TCT CT | SEQ ID NO: 78 |
| IL4I1-F | | TCC GAG GAT GGC TTC TTC TAT C | SEQ ID NO: 79 |
| IL4I1-R | | CGG CTG TAC TGG AGT CTG T | SEQ ID NO: 80 |
| DUSP5-F | | GCT GAC ATT AGC TCC CAC TTT C | SEQ ID NO: 81 |
| DUSP5-R | | GGA ACT GCT TGG TCT TCA TAA | SEQ ID NO: 82 |
| | | GG | |
| TNFRSF18-F | | AGT GGG ACT GCA TGT GTG TC | SEQ ID NO: 83 |
| TNFRSF18-R | | CAA GGT TTG CAG TGG CCT TC | SEQ ID NO: 84 |
| GADD45G- | | CTG CAT GAG TTG CTG CTG TC | SEQ ID NO: 85 |
| Sense | | | |
| GADD45G- | | GCA CTA TGT CGA TGT CGT TCT C | SEQ ID NO: 86 |
| Antisense | | | |
| BATF-F | | GAA GAG TTC AGA GGA GGG AGA | SEQ ID NO: 87 |
| | | A | |
| BATF-R | | GTA GAG CCG CGT TCT GTT TC | SEQ ID NO: 88 |
| LTA-F | | TAC ACC TCC TCC TTC TGG GG | SEQ ID NO: 89 |
| LTA-R | | TCC AAT GAG GTG AGC AGC AG | SEQ ID NO: 90 |
| CDKN1A-F | | TGT GGA CCT GTC ACT GTC TT | SEQ ID NO: 91 |
| CDKN1A-R | | GGC GTT TGG AGT GGT AGA AAT C | SEQ ID NO: 92 |
| IRF8-F | | GGA CAT TTC CGA GCC ATA CAA A | SEQ ID NO: 93 |
| IRF8-R | | ACC GCA CTC CAT CTC TGT AA | SEQ ID NO: 94 |
| PMAIP1-F | | CGG AGA TGC CTG GGA AGA A | SEQ ID NO: 95 |
| PMAIP1-R | | CTC CTG AGT TGA GTA GCA CAC T | SEQ ID NO: 96 |
| Furin F | | AGC GGG ACC TGA ATG TGA AG | SEQ ID NO: 97 |
| Furin R | | CGT GCC TGT TGT CAT TCA TCT | SEQ ID NO: 98 |
| TNF F | | CCT GCT GCA CTT TGG AGT GA | SEQ ID NO: 99 |
| TNF R | | ACA ACA TGG GCT ACA GGC TT | SEQ ID NO: 100 |
| HPRT1-F | | CCC TGG CGT CGT GAT TAG TG | SEQ ID NO: 101 |
| HPRT1-R | | TCG AGC AAG ACG TTC AGT CC | SEQ ID NO: 102 |
| SDHA-F | | CTT GCC AGG ACC TAG AGT TTG T | SEQ ID NO: 103 |
| SDHA-R | | CTC CAC GAC ATC CTT CCG TAA T | SEQ ID NO: 104 |

TABLE 3
Information of Primers and Probes

| | | | | | | | | Primer |
|---|---|---|---|---|---|---|---|---|
| Primer | | | | | | | | final |
| SEQ ID NO: | Length | Start | Stop | Amplicon | Tm | GC % | Location | conc. |
| SEQ ID NO: 1 | 22 | 172 | 194 | 96 | 56.3 | 45 | Exon1 | 125 nM |
| SEQ ID NO: 2 | 23 | 245 | 268 | | 56.6 | 48 | Exon2 | |
| SEQ ID NO: 3 | 22 | 334 | 356 | 87 | 56.5 | | Exon3/4 | 250 nM |
| SEQ ID NO: 4 | 22 | 399 | 421 | | 56.6 | | Exon4 | |
| SEQ ID NO: 5 | 19 | 1938 | 1956 | 219 | 57.4 | 58 | Exon 10 | 125 nM |
| SEQ ID NO: 6 | 17 | 2141 | 2157 | | 59.2 | 65 | Exon 11 | |
| SEQ ID NO: 7 | 21 | 1879 | 1899 | 112 | 56 | 47.62 | Exon10 | 250 nM |
| SEQ ID NO: 8 | 21 | 1990 | 1970 | | 57 | 52.38 | Exon11 | |
| SEQ ID NO: 9 | 20 | 509 | 528 | 124 | 56.6 | 55 | Exon4 | 125 nM |
| SEQ ID NO: 10 | 20 | 632 | 613 | | 56.3 | 55 | Exon5 | |
| SEQ ID NO: 11 | 23 | 2578 | 2601 | 99 | 57 | 52 | Exon24 | 250 nM |
| SEQ ID NO: 12 | 22 | 2655 | 2677 | | 56.2 | 45 | Exon25 | |
| SEQ ID NO: 13 | 20 | 527 | 546 | 112 | 57 | 55 | Exon5 | 250 nM |
| SEQ ID NO: 14 | 20 | 638 | 619 | | 58.3 | 55 | Exon6 | |
| SEQ ID NO: 15 | 21 | 1225 | 1246 | 109 | 56.9 | 48 | Exon7 | 125 nM |
| SEQ ID NO: 16 | 22 | 1312 | 1334 | | 56.7 | 50 | Exon8 | |
| SEQ ID NO: 17 | 21 | 108 | 127 | 94 | 57.1 | 58 | Exon1 | 250 nM |
| SEQ ID NO: 18 | 21 | 182 | 201 | | 57 | 52.4 | Exon2 | |
| SEQ ID NO: 19 | 20 | 904 | 923 | 80 | 56.6 | 50 | Exon10 | 250 nM |
| SEQ ID NO: 20 | 20 | 964 | 983 | | 57 | 55 | Exon11 | |
| SEQ ID NO: 21 | 20 | 369 | 388 | 112 | 57.5 | 60 | Exon3 | 250 nM |
| SEQ ID NO: 22 | 20 | 461 | 480 | | 57.3 | 55 | Exon4 | |
| SEQ ID NO: 23 | 20 | 870 | 889 | 175 | 57.4 | 55 | Exon6/7 | 250 nM |
| SEQ ID NO: 24 | 20 | 1025 | 1044 | | 57 | 55 | Exon8 | |
| SEQ ID NO: 25 | 20 | 385 | 404 | 188 | 57.4 | 55 | Exon2 | 250 nM |
| SEQ ID NO: 26 | 20 | 553 | 572 | | 57.3 | 55 | Exon3 | |
| SEQ ID NO: 27 | 22 | 844 | 865 | 139 | 57.1 | 54.5 | Exon 8 | 250 nM |
| SEQ ID NO: 28 | 20 | 963 | 982 | | 56.9 | 50 | Exon 9 | |
| SEQ ID NO: 29 | 20 | 772 | 791 | 177 | 57.1 | 55 | Exon7 | 250 nM |
| SEQ ID NO: 30 | 20 | 929 | 948 | | 57.3 | 55 | Exon9 | |
| SEQ ID NO: 31 | 20 | 2236 | 2255 | 173 | 57.6 | 60 | Exon10/11 | 250 nM |
| SEQ ID NO: 32 | 20 | 2389 | 2408 | | 57.2 | 55 | Exon12 | |
| SEQ ID NO: 33 | 20 | 1770 | 1789 | 143 | 57.2 | 55 | Exon7 | 250 nM |
| SEQ ID NO: 34 | 20 | 1893 | 1912 | | 57.4 | 55 | Exon7/8 | |
| SEQ ID NO: 35 | 21 | 1163 | 1184 | 117 | 56.4 | 48 | Exon 4/5 | 250 nM |
| SEQ ID NO: 36 | 22 | 1258 | 1280 | | 57 | 55 | Exon 6 | |
| SEQ ID NO: 37 | 23 | 191 | 213 | 90 | 56.6 | 48 | Exon2 | 250 nM |
| SEQ ID NO: 38 | 22 | 260 | 281 | | 57 | 50 | Exon3 | |
| SEQ ID NO: 39 | 21 | 770 | 791 | 105 | 57.3 | 47.6 | Exon 4/5 | 250 nM |
| SEQ ID NO: 40 | 21 | 854 | 875 | | 57.7 | 57.1 | Exon 6 | |
| SEQ ID NO: 41 | 19 | 1153 | 1171 | 193 | 56.3 | 53 | Exon7 | 62.5 nM |
| SEQ ID NO: 42 | 19 | 1327 | 1345 | | 57.2 | 58 | Exon7/8 | |
| SEQ ID NO: 43 | 17 | 390 | 406 | 75 | 59 | 71 | Exon3 | 500 nM |
| SEQ ID NO: 44 | 23 | 442 | 464 | | 56.3 | 44 | Exon3/4 | |
| SEQ ID NO: 45 | 22 | 172 | 193 | 67 | 56.9 | 50 | Exon 1 | 250 nM |
| SEQ ID NO: 46 | 20 | 219 | 238 | | 57.2 | 55 | Exon2/3 | |
| SEQ ID NO: 47 | 20 | | | 153 | | | | 250 nM |
| SEQ ID NO: 48 | 20 | | | | | | | |

### Example 3

### Methods and Materials

### A. RNA Sequencing and Analysis

Samples were prepared with the Illumina TruSeq RNA library preparation kit and sequenced on the Illumina GA IIx.

RNA sequencing data were prescreened: all failed probe data were removed and all genes with fewer than 3 samples (out of 12) with FPKM>5 were removed. Total 8695 genes passed prescreen (out of 24789).

RNA sequencing data were analyzed using BRB Array Tools developed by Richard Simon & BRB-ArrayTools Development Team. It is available at the website of Biometric Research Branch, Division of Cancer Treatment and Diagnosis, National Cancer Institute. BRB-ArrayTools is an integrated package for the visualization and statistical analysis of DNA microarray gene expression data. It was developed by professional statisticians experienced in the analysis of microarray data and involved in the development of improved methods for the design and analysis of microarray based experiments. The array tools package utilizes an Excel front end. Scientists are familiar with Excel and utilizing Excel as the front end makes the system portable and not tied to any database. The input data is assumed to be in the form of Excel spreadsheets describing the expression values and a spreadsheet providing user-specified phenotypes for the samples arrayed. The analytic and visualization tools are integrated into Excel as an add-in. The analytic and visualization tools themselves are developed in the powerful R statistical system, in C and Fortran programs and in Java applications. Visual Basic for Applications is the glue that integrates the components and hides the complexity of the analytic methods from the user. The system incorporates a variety of powerful analytic and visualization tools developed specifically for microarray data analysis. In one example, genes with highest 20% variance were selected and genes missing >50% values were eliminated.

### B. Real-Time PCR

Fluidigm qPCR technology was used. In one example, PCR was performed in 48×48 format according to protocol with modification of primer concentrations which were adjusted to optimal concentrations as noted in Table 3.

### C. Cell Isolation and Culture

PBMC (peripheral blood mononuclear cells) were isolated from healthy volunteers by separation on Ficoll-Hypaque gradients. CD4⁺ T cells were isolated according to manufacturer's recommendations using negative selection by depletion with magnetic beads (Stemcell Technologies, Vancouver, BC, Canada) and were at least 95% pure.

CD4⁺ T cells were cultured overnight (37° C. with 5% CO2) in RPMI 1640 with 10% fetal calf serum. For the primed group (IL12+IL18), cells were treated with IL-12 (0.5 ng/ml) and IL-18 (50 ng/ml) for 8 hours at 37° C. prior to RNA isolation. For the TL1A-stimulated group (IL12+IL18+TL1A), cells were treated with IL-12 (0.5 ng/ml), IL-18 (50 ng/ml), and recombinant TL1A (100 ng/ml) (Fitzgerald, North Acton, Mass.) for 8 hours at 37° C. prior to RNA isolation. RNA was isolated using RNeasy Plus Mini Kit (Qiagen, Germantown, Md.). Other examples are described in Papadakis et al. (TL1A synergizes with IL-12 and IL-18 to enhance IFN-gamma production in human T cells and NK cells; J Immunol. 2004 Jun. 1; 172(11):7002-7), which is incorporated herein by reference as though fully set forth.

Other examples are described in Papadakis et al. (TL1A synergizes with IL-12 and IL-18 to enhance IFN-gamma production in human T cells and NK cells; J Immunol. 2004 Jun. 1; 172(11):7002-7), which is incorporated herein by reference as though fully set forth.

### Example 4

TABLE 4
Top TL1A Response Genes from IFN-γ-Secreting Cells

| Gene | p-value | FDR |
|---|---|---|
| BATF | <1e−07 | <1e−07 |
| CCL20 | 2.00E−07 | 1.33E−06 |
| CD274 | <1e−07 | <1e−07 |
| CD83 | <1e−07 | <1e−07 |
| CDKN1A | 3.20E−06 | 1.17E−05 |
| CHAC1 | <1e−07 | <1e−07 |
| CSF2 | 9.00E−07 | 4.30E−06 |
| DUSP5 | <1e−07 | <1e−07 |
| FEZ1 | 4.00E−07 | 2.28E−06 |
| GADD45G | 1.00E−07 | 7.67E−07 |
| HMSD | 3.63E−04 | 6.50E−04 |
| IFNG | <1e−07 | <1e−07 |
| IL22 | <1e−07 | <1e−07 |
| IL26 | 2.00E−07 | 1.33E−06 |
| IL4I1 | 1.20E−05 | 3.45E−05 |
| IRF8 | 3.02E−05 | 7.42E−05 |
| LTA | <1e−07 | <1e−07 |
| MFSD2A | 1.23E−05 | 3.52E−05 |
| MYO1B | <1e−07 | <1e−07 |
| NFKBIA | <1e−07 | <1e−07 |
| RPL21 | <1e−07 | <1e−07 |
| SGK1 | <1e−07 | <1e−07 |
| TNFRSF18 | 9.00E−07 | 4.30E−06 |
| TNFRSF4 | 4.64E−05 | 1.07E−04 |
| TRAF4 | <1e−07 | <1e−07 |
| XIST | 1.30E−04 | 2.62E−04 |

### Example 5

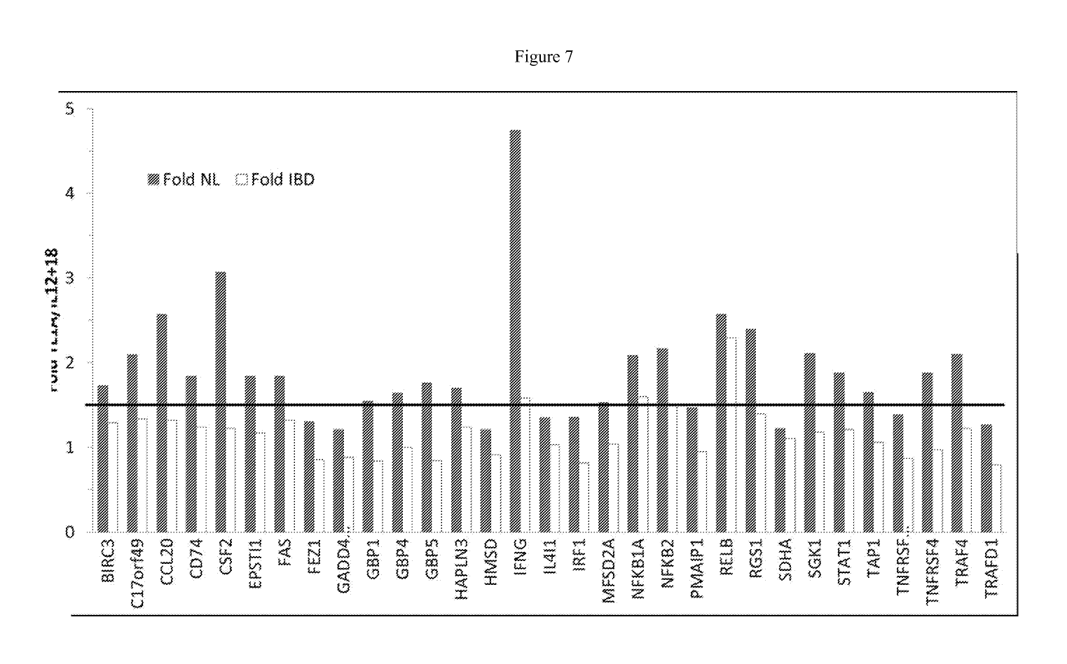

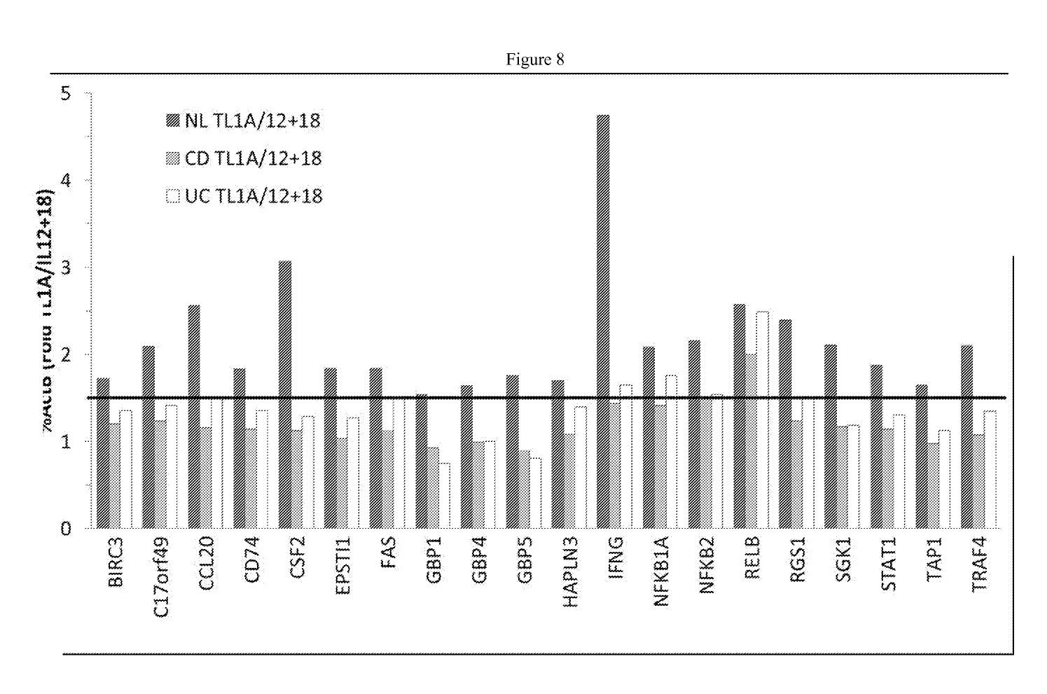

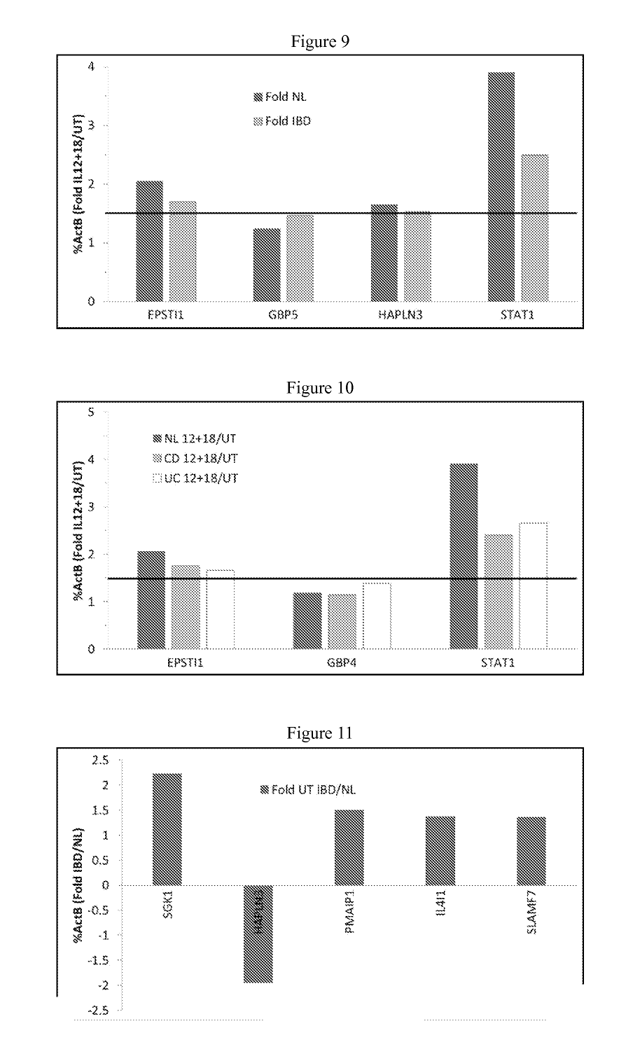

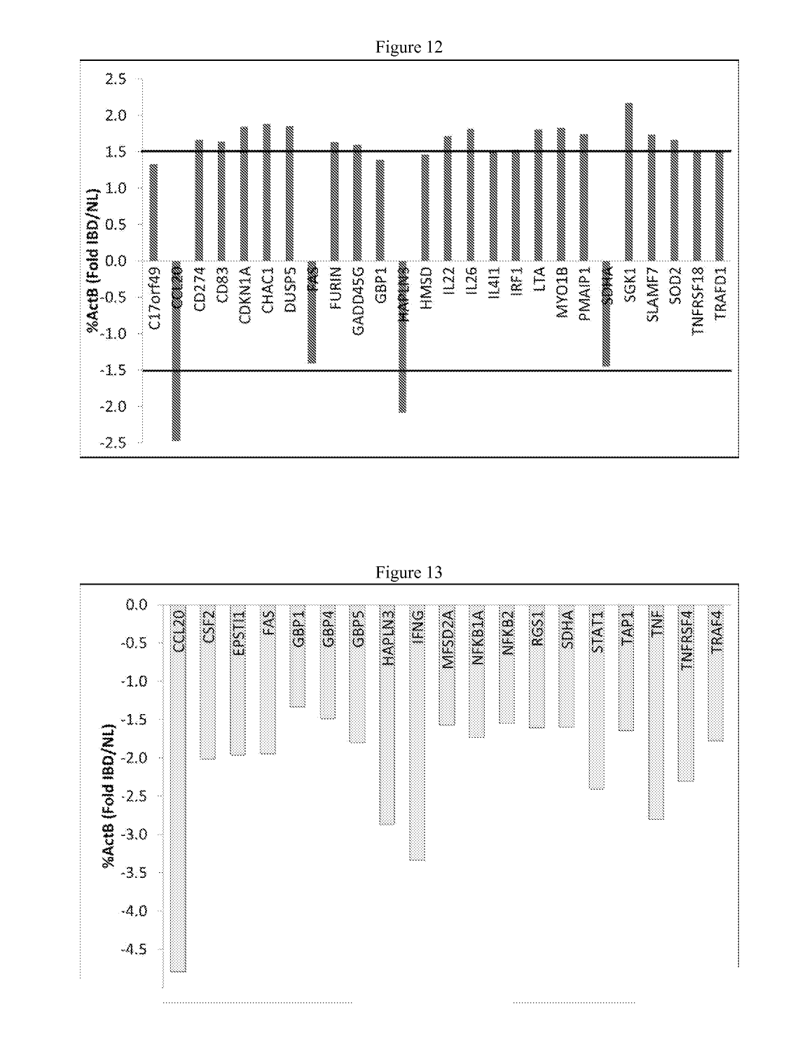

In one example, 20 normal control (NL), 20 CD, and 18 UC samples were rested overnight, activated for 8 hours with (IL12+IL18) or (IL12+IL18/TL1A), and analyzed for expression levels of 48 genes. In another example, 21 NL, 15 NL-H, 20 CD, and 18 UC samples were rested overnight, activated for 8 hours with (IL12+IL18) or (IL12+IL18+TL1A), and analyzed for expression levels of 20 genes. Results are shown in FIGS. 7-13 and Table 5.

TABLE 5
Expression Levels Differ in IBD vs. NL (p values)

| GENE | UT | 12 + 18 | TL1A |
|---|---|---|---|
| C17orf49 | | Upregulated (0.0149) | |
| CCL20 | | Downregulated (0.0479) | Downregulated (0.0015) |
| CD274 | | Upregulated (0.0228) | |
| CD83 | | Upregulated (0.004) | |
| CDKN1A | | Upregulated (0.0296) | |
| CHAC1 | | Upregulated (0.0033) | |
| CSF2 | | | Downregulated (0.0219) |
| DUSP5 | | Upregulated (0.027) | |
| EPSTI1 | | | Downregulated (0.0036) |
| FAS | | Downregulated (0.0049) | Downregulated (0.0004) |
| FURIN | | Upregulated (0.0473) | |
| GADD45G | | Upregulated (0.0397) | |
| GBP1 | | Upregulated (0.0359) | Downregulated (0.0234) |
| GBP4 | | | Downregulated (0.0322) |
| GBP5 | | | Downregulated (0.0049) |
| HAPLN3 | Downregulated (0.0004) | Downregulated (0.0002) | Downregulated (0.0001) |
| HMSD | | Upregulated (0.0236) | |
| IFNG | | | Downregulated (0.0419) |
| IL22 | | Upregulated (0.0115) | |
| IL26 | | Upregulated (0.0197) | |
| IL4I1 | Upregulated (0.0126) | Upregulated (0.003) | |
| IRF1 | | Upregulated (0.0229) | |
| LTA | | Upregulated (0.0119) | |
| MFSD2A | | | Downregulated (0.044) |
| MYO1B | | Upregulated (0.0218) | |
| NFKB1A | | | Downregulated (0.0434) |
| NFKB2 | | | Downregulated (0.0097) |
| PMAIP1 | Upregulated (0.0045) | Upregulated (0.0016) | |
| RGS1 | | | Downregulated (0.0066) |
| SGK1 | Upregulated (0.0001) | Upregulated (0.0002) | |
| SLAMF7 | Upregulated (0.0342) | Upregulated (0.0023) | |
| SOD2 | | Upregulated (0.0163) | |
| STAT1 | | | Downregulated (0.0029) |
| TAP1 | | | Downregulated (0.0297) |
| TNF | | | Downregulated (0.0428) |
| TNFRSF18 | | Upregulated (0.0283) | |
| TNFRSF4 | | | Downregulated (0.018) |
| TRAF4 | | | Downregulated (0.0013) |
| TRAFD1 | | Upregulated (0.0097) | |

### Example 6

TABLE 6
List of Genes

| | | | | |
|---|---|---|---|---|
| ACTB | DUSP5 | HAPLN3 | MFSD2A | SLAMF7 |
| BATF | EEF1A1 | HMSD | MYO1B | SLC7A5 |
| BIRC3 | EPSTI1 | HPRT1 | NFKB1A | SOD2 |
| C17orf49 | FAS | IFNG | NFKB2 | STAT1 |
| CCL20 | FEZ1 | IL22 | NFKBIA | TAP1 |
| CD274 | Furin | IL26 | PMAIP1 | TNF |
| CD74 | GADD45B | IL4I1 | RELB | TNFRSF18 |
| CD83 | GADD45G | IRF1 | RGS1 | TNFRSF4 |
| CDKN1A | GBP1 | IRF4 | RPL21 | TRAF4 |
| CHAC1 | GBP4 | IRF8 | SDHA | TRAFD1 |
| CSF2 | GBP5 | LTA | SGK1 | XIST |

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit to the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.)

1. A method of selecting a treatment for a subject, comprising:
obtaining a sample from the subject;
assaying the expression level of one or more biomarkers associated with TL1A signaling in the sample;
comparing the expression level to a reference value of expression level of the one or more biomarkers associated with TL1A signaling; and
prescribing an anti-TL1A therapy to the subject if the subject has a high expression level relative to the reference value of one or more biomarkers associated with TL1A signaling, or prescribing no anti-TL1A therapy to the subject if the subject does not have a high expression level relative to the reference value of one or more biomarkers associated with TL1A signaling.

2. The method of claim 1, further comprising stimulating the sample with IL12, IL18, or TL1A, or a combination thereof, before assaying the expression level of one or more biomarkers associated with TL1A signaling in the sample.

3. The method of claim 1, wherein the one or more biomarkers associated with TL1A signaling is listed in Table 1, Table 4, Table 5 and/or Table 6 herein.

4. The method of claim 1, wherein the one or more biomarkers associated with TL1A signaling is selected from the group consisting of BIRC3, C17orf49, CCL20, CSF2, CD274, CD74, EPSTI1, FAS, GBP1, GBP4, GBP5, HAPLN3, IFNG, IRF1, NFKBIA, NFKB2, RELB, RGS1, SGK1, STAT1, TAP1, and TRAFD1.

5. The method of claim 1, wherein the one or more biomarkers associated with TL1A signaling is selected from the group consisting of BATF, CCL20, CD274, CD83, CDKN1A, CHAC1, CSF2, DUSP5, FEZ1, GADD45G, HMSD, IFNG, IL22, IL26, IL411, IRF8, LTA, MFSD2A, MYO1B, NFKBIA, RPL21, SGK1, TNFRSF18, TNFRSF4, TRAF4, and XIST.

6. The process of claim 1, wherein the subject is human.

7. The method of claim 1, wherein the subject has a symptom of a TL1A-associated disease, or is suspected of having a TL1A-associated disease, or is diagnosed with a TL1A-associated disease.

8. (canceled)

9. (canceled)

10. The method of claim 1, wherein the sample comprises a T cell, CD4⁺ T cell, CD8⁺ T cell, CD56⁺ T cell, CD45R0⁺ T cell, CD45RA⁺ T cell, NK cell, peripheral blood mononuclear cell (PBMC), or peripheral blood lymphocyte (PBL), or a combination thereof.

11. The method of claim 7, wherein the TL1A associated disease is fibrosis, Crohn's disease (CD), inflammatory bowel disease (IBD), chronic obstructive pulmonary disease, allergic lung inflammation, asthma, atherosclerosis, lupus, rheumatoid arthritis (RA), multiple sclerosis (MS), psoriasis, type 1 diabetes, lung carcinoma, colon carcinoma, leukemia, lymphoma, transplant rejection, graft versus host disease, or central nervous system injury.

12. The method of claim 2, wherein assaying the expression level of one or more genes listed in Table 1, Table 4, Table 5 and/or Table 6 in the sample comprises assaying an mRNA level.

13. The method of claim 12, wherein assaying an mRNA level comprises
using RNA sequencing, northern blot, in situ hybridization, hybridization array, serial analysis of gene expression (SAGE), reverse transcription PCR, real-time PCR, real-time reverse transcription PCR, or quantitative PCR, or a combination thereof, or
contacting the sample with a polynucleotide probe capable of specifically hybridizing to mRNA of one or more genes listed in Table 1, Table 4, Table 5 and/or Table 6 and thereby forming a probe-target hybridization complex, or
contacting the sample with one or more polynucleotide primers capable of specifically hybridizing to mRNAs of genes listed in Table 1, Table 4, Table 5 and/or Table 6, forming a primer-template hybridization complex, and performing a PCR reaction.

14. (canceled)

15. (canceled)

16. (canceled)

17. The method of claim 1, wherein assaying the expression level of one or more genes listed in Table 1, Table 4, Table 5 and/or Table 6 in the sample comprises assaying a protein level.

18. The method of claim 17, wherein assaying a protein level comprises
using western blot, enzyme-linked immunosorbent assay (ELISA), radioimmunoassay, or mass spectrometry, or a combination thereof, or
contacting the sample with antibodies capable of specifically binding to proteins of genes listed in Table 1, Table 4, Table 5 and/or Table 6 and thereby forming antigen-antibody complexes.

19. (canceled)

20. The method of claim 1, wherein the reference value of expression level is
the median or mean expression level from a population of subjects who have no TL1A-associated disease, or
the median or mean expression level from a population of subjects who are unlikely to be responsive to an anti-TL1A therapy, or
the median or mean expression level from a population of subjects who are not responsive to an anti-TL1A therapy.

21. (canceled)

22. (canceled)

23. The method of claim 1, wherein the anti-TL1A therapy comprises
an anti-TL1A antibody or a fragment thereof, or
an anti-DR3 antibody or a fragment thereof, or
soluble decoy DR3 polypeptide, a polypeptide comprising a DR3 extracellular domain, or a polypeptide comprising a DR3 pre-ligand assembly domain, or a combination thereof, or
nucleic acid antagonist of TL1A, or a nucleic acid antagonist of DR3, or a combination thereof, or
a GEP peptide, Atsttrin or a variant thereof, or a combination thereof.

24. (canceled)

25. (canceled)

26. (canceled)

27. (canceled)

28. (canceled)

29. (canceled)

30. (canceled)

31. (canceled)

32. (canceled)

33. A method of treating a subject, comprising:
obtaining a sample from the subject;
assaying the expression level of one or more genes listed in Table 1, Table 4, Table 5 and/or Table 6 in the sample;
comparing the expression level to a reference value of expression level of the one or more genes listed in Table 1, Table 4, Table 5 and/or Table 6; and
administering an anti-TL1A therapy to the subject if the subject has a high expression level relative to the reference value of one or more genes listed in Table 1, Table 4, Table 5 and/or Table 6, or administering no anti-TL1A therapy to the subject if the subject does not have a high expression level relative to the reference value of any of the genes listed in Table 1, Table 4, Table 5 and/or Table 6.

34. The method of claim 33, wherein the one or more genes is selected from the group consisting of BIRC3, C17orf49, CCL20, CSF2, CD274, CD74, EPSTI1, FAS, GBP1, GBP4, GBP5, HAPLN3, IFNG, IRF1, NFKBIA, NFKB2, RELB, RGS1, SGK1, STAT1, TAP1, and TRAFD1.

35. The method of claim 33, wherein the one or more genes is selected from the group consisting of BATF, CCL20, CD274, CD83, CDKN1A, CHAC1, CSF2, DUSP5, FEZ1, GADD45G, HMSD, IFNG, IL22, IL26, IL411, IRF8, LTA, MFSD2A, MYO1B, NFKBIA, RPL21, SGK1, TNFRSF18, TNFRSF4, TRAF4, and XIST.

36. A method, comprising:
obtaining a sample from a subject;
assaying the expression level of one or more genes listed in Table 1, Table 4, Table 5 and/or Table 6 herein in the sample;
comparing the expression level to a reference value of expression level of the one or more genes; and
diagnosing a disease in the subject according to the relative difference between the expression level and the reference value.

37. The method of claim 36, further comprising stimulating the sample with IL12, IL18, or TL1A, or a combination thereof, before assaying the expression level of one or more genes in the sample.

38. The method of claim 36, further comprising diagnosing the disease in the subject if the subject has an expression level higher than the reference value, or not diagnosing the disease in the subject if the subject does not have an expression level higher than the reference value.

39. The method of claim 36, further comprising diagnosing the disease in the subject if the subject has an expression level lower than the reference value, or not diagnosing the disease subtype in the subject if the subject does not have an expression level lower than the reference value.

40. The method of claim 36, wherein the disease is a TL1A-associated disease.

41. The method of claim 36, wherein the disease is fibrosis, Crohn's disease (CD), inflammatory bowel disease (IBD), chronic obstructive pulmonary disease, allergic lung inflammation, asthma, atherosclerosis, lupus, rheumatoid arthritis (RA), multiple sclerosis (MS), psoriasis, type 1 diabetes, lung carcinoma, colon carcinoma, leukemia, lymphoma, transplant rejection, graft versus host disease, or central nervous system injury.

42. The method of claim 36, wherein the disease is an IBD subtype responsive to an anti-TL1A therapy.

43. The method of claim 36, wherein the subject is a human.

44. The method of claim 36, wherein the subject has a symptom of an IBD subtype, or is suspected of having an IBD subtype.

45. (canceled)

46. The method of claim 36, wherein the one or more genes is selected from the group consisting of BIRC3, C17orf49, CCL20, CSF2, CD274, CD74, EPSTI1, FAS, GBP1, GBP4, GBP5, HAPLN3, IFNG, IRF1, NFKBIA, NFKB2, RELB, RGS1, SGK1, STAT1, TAP1, and TRAFD1.

47. The method of claim 36, wherein the one or more genes is selected from the group consisting of BATF, CCL20, CD274, CD83, CDKN1A, CHAC1, CSF2, DUSP5, FEZ1, GADD45G, HMSD, IFNG, IL22, IL26, IL411, IRF8, LTA, MFSD2A, MYO1B, NFKBIA, RPL21, SGK1, TNFRSF18, TNFRSF4, TRAF4, and XIST.

48. The method of claim 36, wherein the sample comprises a T cell, CD4⁺ T cell, CD8⁺ T cell, CD56⁺ T cell, CD45R0⁺ T cell, CD45RA⁺ T cell, NK cell, peripheral blood mononuclear cell (PBMC), or peripheral blood lymphocyte (PBL), or a combination thereof.

49. (canceled)

50. (canceled)


SEQUENCE LISTING (104 sequences)

SEQ ID NO: 1 - DNA, 22 bp, Artificial Sequence
  synthetic
ttgggttctcttggctgttact

SEQ ID NO: 2 - DNA, 23 bp, Artificial Sequence
  synthetic
atccgctacatctgaatgacctg

SEQ ID NO: 3 - DNA, 22 bp, Artificial Sequence
  synthetic
cttgccaaccaaactggtcaaa

SEQ ID NO: 4 - DNA, 22 bp, Artificial Sequence
  synthetic
acaagccagccagaactcaata

SEQ ID NO: 5 - DNA, 19 bp, Artificial Sequence
  synthetic
ggttggcggcgattcaaag

SEQ ID NO: 6 - DNA, 17 bp, Artificial Sequence
  synthetic
agtcctctgggcgtgct

SEQ ID NO: 7 - DNA, 21 bp, Artificial Sequence
  synthetic
acttcaggaacaggagcaact

SEQ ID NO: 8 - DNA, 21 bp, Artificial Sequence
  synthetic
ggtacatgcctttcgtcgtct

SEQ ID NO: 9 - DNA, 20 bp, Artificial Sequence
  synthetic
aggaggtgaaagaccagagc

SEQ ID NO: 10 - DNA, 20 bp, Artificial Sequence
  synthetic
ctcttagcatctcggctgga

SEQ ID NO: 11 - DNA, 23 bp, Artificial Sequence
  synthetic
gtttgacgaggtgtctcggatag

SEQ ID NO: 12 - DNA, 22 bp, Artificial Sequence
  synthetic
aactgtcgccagagaagatgaa

SEQ ID NO: 13 - DNA, 20 bp, Artificial Sequence
  synthetic
tgctctgaacgactccgatg

SEQ ID NO: 14 - DNA, 20 bp, Artificial Sequence
  synthetic
agaatccagggtcaggctgt

SEQ ID NO: 15 - DNA, 21 bp, Artificial Sequence
  synthetic
agcaagacacaaggcagaaga

SEQ ID NO: 16 - DNA, 22 bp, Artificial Sequence
  synthetic
cagaacattccgctccgacata

SEQ ID NO: 17 - DNA, 21 bp, Artificial Sequence
  synthetic
ggacagagcaatgggtgagag

SEQ ID NO: 18 - DNA, 21 bp, Artificial Sequence
  synthetic
actagacaaatgggggccatc

SEQ ID NO: 19 - DNA, 20 bp, Artificial Sequence
  synthetic
gcagcagcaagagcaagaaa

SEQ ID NO: 20 - DNA, 20 bp, Artificial Sequence
  synthetic
ggagtcggtccagaaaagca

SEQ ID NO: 21 - DNA, 20 bp, Artificial Sequence
  synthetic
gttgaaggaccagctctccc

SEQ ID NO: 22 - DNA, 20 bp, Artificial Sequence
  synthetic
cttgtagtcggcaccaccat

SEQ ID NO: 23 - DNA, 20 bp, Artificial Sequence
  synthetic
attgacccctacaacgctgg

SEQ ID NO: 24 - DNA, 20 bp, Artificial Sequence
  synthetic
tccgcagctctgatgtgttt

SEQ ID NO: 25 - DNA, 20 bp, Artificial Sequence
  synthetic
aagtgatccgccaggtgaag

SEQ ID NO: 26 - DNA, 20 bp, Artificial Sequence
  synthetic
ctgctcacaggcaaggtgta

SEQ ID NO: 27 - DNA, 22 bp, Artificial Sequence
  synthetic
cgcttctctgccttccttagag

SEQ ID NO: 28 - DNA, 20 bp, Artificial Sequence
  synthetic
agcctgctgtcttgtccatt

SEQ ID NO: 29 - DNA, 20 bp, Artificial Sequence
  synthetic
gcactccttggagcaaaagc

SEQ ID NO: 30 - DNA, 20 bp, Artificial Sequence
  synthetic
aagacacaccagcagtagcc

SEQ ID NO: 31 - DNA, 20 bp, Artificial Sequence
  synthetic
gtggtaggagtgaaggtggc

SEQ ID NO: 32 - DNA, 20 bp, Artificial Sequence
  synthetic
gcacagaagacaggaaccga

SEQ ID NO: 33 - DNA, 20 bp, Artificial Sequence
  synthetic
tttgagtacctggaccgcac

SEQ ID NO: 34 - DNA, 20 bp, Artificial Sequence
  synthetic
aatgtcagcccctgtagcac

SEQ ID NO: 35 - DNA, 21 bp, Artificial Sequence
  synthetic
agtggtttccaaggtgtgagt

SEQ ID NO: 36 - DNA, 22 bp, Artificial Sequence
  synthetic
ctgggctgtctgatgtggatag

SEQ ID NO: 37 - DNA, 23 bp, Artificial Sequence
  synthetic
caacggcttctactactccaaca

SEQ ID NO: 38 - DNA, 22 bp, Artificial Sequence
  synthetic
caccaccagcttcactccatta

SEQ ID NO: 39 - DNA, 21 bp, Artificial Sequence
  synthetic
actgtgacccttgcaccaaat

SEQ ID NO: 40 - DNA, 21 bp, Artificial Sequence
  synthetic
gccaccccaagttagatctgg

SEQ ID NO: 41 - DNA, 19 bp, Artificial Sequence
  synthetic
cacacggctcacattgcat

SEQ ID NO: 42 - DNA, 19 bp, Artificial Sequence
  synthetic
cacgaacagcaaagcgacc

SEQ ID NO: 43 - DNA, 17 bp, Artificial Sequence
  synthetic
cgtgctgctgaccgagg

SEQ ID NO: 44 - DNA, 23 bp, Artificial Sequence
  synthetic
aaggtctcaaacatgatctgggt

SEQ ID NO: 45 - DNA, 22 bp, Artificial Sequence
  synthetic
gagacactgctgctgagatgaa

SEQ ID NO: 46 - DNA, 20 bp, Artificial Sequence
  synthetic
ggctcctggaggtcaaacat

SEQ ID NO: 47 - DNA, 20 bp, Artificial Sequence
  synthetic
gtctgtgtgcgcaaatccaa

SEQ ID NO: 48 - DNA, 20 bp, Artificial Sequence
  synthetic
gaaacctccaaccccagcaa

SEQ ID NO: 49 - DNA, 20 bp, Artificial Sequence
  synthetic
tgaatgtggacccagacagc

SEQ ID NO: 50 - DNA, 20 bp, Artificial Sequence
  synthetic
actggatgagcgtgaagtgg

SEQ ID NO: 51 - DNA, 22 bp, Artificial Sequence
  synthetic
ggagcagagagaaccctatgaa

SEQ ID NO: 52 - DNA, 21 bp, Artificial Sequence
  synthetic
aggtgaagaggaagccagtaa

SEQ ID NO: 53 - DNA, 21 bp, Artificial Sequence
  synthetic
cgtgatctacctgcacacttg

SEQ ID NO: 54 - DNA, 22 bp, Artificial Sequence
  synthetic
acataccctcatggctctcata

SEQ ID NO: 55 - DNA, 22 bp, Artificial Sequence
  synthetic
tgtaacctcagaagtgggagtg

SEQ ID NO: 56 - DNA, 20 bp, Artificial Sequence
  synthetic
acagtcaactccaggcttgt

SEQ ID NO: 57 - DNA, 20 bp, Artificial Sequence
  synthetic
ccaacatgcctcaccctcat

SEQ ID NO: 58 - DNA, 20 bp, Artificial Sequence
  synthetic
ggaaccgaccagctctttca

SEQ ID NO: 59 - DNA, 20 bp, Artificial Sequence
  synthetic
ctcttcctgatcgccgtctc

SEQ ID NO: 60 - DNA, 20 bp, Artificial Sequence
  synthetic
gaccacctgcatgagcttct

SEQ ID NO: 61 - DNA, 20 bp, Artificial Sequence
  synthetic
cactgcaaggaacaacaggc

SEQ ID NO: 62 - DNA, 20 bp, Artificial Sequence
  synthetic
tgctcccacacatcaatccc

SEQ ID NO: 63 - DNA, 20 bp, Artificial Sequence
  synthetic
gcccagcaggttcacaacta

SEQ ID NO: 64 - DNA, 20 bp, Artificial Sequence
  synthetic
aggtggggcacaagcataaa

SEQ ID NO: 65 - DNA, 20 bp, Artificial Sequence
  synthetic
tggggagaaactgttccacg

SEQ ID NO: 66 - DNA, 20 bp, Artificial Sequence
  synthetic
tgtgcttagcctgttgctga

SEQ ID NO: 67 - DNA, 20 bp, Artificial Sequence
  synthetic
aacgattccagaagaccgca

SEQ ID NO: 68 - DNA, 20 bp, Artificial Sequence
  synthetic
gaaagtcctccacaaagcgt

SEQ ID NO: 69 - DNA, 23 bp, Artificial Sequence
  synthetic
gctcacagcagatcaggtaattg

SEQ ID NO: 70 - DNA, 23 bp, Artificial Sequence
  synthetic
tttctcctccatcctcttcttcc

SEQ ID NO: 71 - DNA, 20 bp, Artificial Sequence
  synthetic
gcagggagacaccttccatc

SEQ ID NO: 72 - DNA, 20 bp, Artificial Sequence
  synthetic
tcttcaaggagcgtcaccac

SEQ ID NO: 73 - DNA, 21 bp, Artificial Sequence
  synthetic
accgtgctaatgggaagagta

SEQ ID NO: 74 - DNA, 22 bp, Artificial Sequence
  synthetic
caccaatggaacctctgacttg

SEQ ID NO: 75 - DNA, 20 bp, Artificial Sequence
  synthetic
caacgggctctttggagaaa

SEQ ID NO: 76 - DNA, 23 bp, Artificial Sequence
  synthetic
cgtgttgtggacttctctgtatc

SEQ ID NO: 77 - DNA, 20 bp, Artificial Sequence
  synthetic
cgaagatgtggacttgccct

SEQ ID NO: 78 - DNA, 20 bp, Artificial Sequence
  synthetic
ggggtgtctccatcctctct

SEQ ID NO: 79 - DNA, 22 bp, Artificial Sequence
  synthetic
tccgaggatggcttcttctatc

SEQ ID NO: 80 - DNA, 19 bp, Artificial Sequence
  synthetic
cggctgtactggagtctgt

SEQ ID NO: 81 - DNA, 22 bp, Artificial Sequence
  synthetic
gctgacattagctcccactttc

SEQ ID NO: 82 - DNA, 23 bp, Artificial Sequence
  synthetic
ggaactgcttggtcttcataagg

SEQ ID NO: 83 - DNA, 20 bp, Artificial Sequence
  synthetic
agtgggactgcatgtgtgtc

SEQ ID NO: 84 - DNA, 20 bp, Artificial Sequence
  synthetic
caaggtttgcagtggccttc

SEQ ID NO: 85 - DNA, 20 bp, Artificial Sequence
  synthetic
ctgcatgagttgctgctgtc

SEQ ID NO: 86 - DNA, 22 bp, Artificial Sequence
  synthetic
gcactatgtcgatgtcgttctc

SEQ ID NO: 87 - DNA, 22 bp, Artificial Sequence
  synthetic
gaagagttcagaggagggagaa

SEQ ID NO: 88 - DNA, 20 bp, Artificial Sequence
  synthetic
gtagagccgcgttctgtttc

SEQ ID NO: 89 - DNA, 20 bp, Artificial Sequence
  synthetic
tacacctcctccttctgggg

SEQ ID NO: 90 - DNA, 20 bp, Artificial Sequence
  synthetic
tccaatgaggtgagcagcag

SEQ ID NO: 91 - DNA, 20 bp, Artificial Sequence
  synthetic
tgtggacctgtcactgtctt

SEQ ID NO: 92 - DNA, 22 bp, Artificial Sequence
  synthetic
ggcgtttggagtggtagaaatc

SEQ ID NO: 93 - DNA, 22 bp, Artificial Sequence
  synthetic
ggacatttccgagccatacaaa

SEQ ID NO: 94 - DNA, 20 bp, Artificial Sequence
  synthetic
accgcactccatctctgtaa

SEQ ID NO: 95 - DNA, 19 bp, Artificial Sequence
  synthetic
cggagatgcctgggaagaa

SEQ ID NO: 96 - DNA, 22 bp, Artificial Sequence
  synthetic
ctcctgagttgagtagcacact

SEQ ID NO: 97 - DNA, 20 bp, Artificial Sequence
  synthetic
agcgggacctgaatgtgaag

SEQ ID NO: 98 - DNA, 21 bp, Artificial Sequence
  synthetic
cgtgcctgttgtcattcatct

SEQ ID NO: 99 - DNA, 20 bp, Artificial Sequence
  synthetic
cctgctgcactttggagtga

SEQ ID NO: 100 - DNA, 20 bp, Artificial Sequence
  synthetic
acaacatgggctacaggctt

SEQ ID NO: 101 - DNA, 20 bp, Artificial Sequence
  synthetic
ccctggcgtcgtgattagtg

SEQ ID NO: 102 - DNA, 20 bp, Artificial Sequence
  synthetic
tcgagcaagacgttcagtcc

SEQ ID NO: 103 - DNA, 22 bp, Artificial Sequence
  synthetic
cttgccaggacctagagtttgt

SEQ ID NO: 104 - DNA, 22 bp, Artificial Sequence
  synthetic
ctccacgacatccttccgtaat